SEQUENCE LISTING (32 sequences)

SEQ ID NO: 1 - DNA, 39 bp, Artificial Sequence
  Pptb-BsaI-F
tacgggtctcgtccataagtcagcagaaagtataatgag

SEQ ID NO: 2 - DNA, 50 bp, Artificial Sequence
  RBD-BsaI-R
tataggtctctgtctttaaaagtttacgcatttgttcttcactagattag

SEQ ID NO: 3 - DNA, 44 bp, Artificial Sequence
  Lig-BsaI-R
tataggtctctgtctttatggacgttgtggagttgaatgatatg

SEQ ID NO: 4 - DNA, 42 bp, Artificial Sequence
  Pfdx-BsaI-F
tacgggtctcgtccagtgtagtagcctgcgaaataagtaagg

SEQ ID NO: 5 - DNA, 41 bp, Artificial Sequence
  NC-BsaI-R
tataggtctctgtctttaggcctgagttgagtcagcactgc

SEQ ID NO: 6 - DNA, 22 bp, Artificial Sequence
  pGGv2-R
ctgttgaaccattagccgtctc

SEQ ID NO: 7 - DNA, 17 bp, Artificial Sequence
  pGGv2-F
ccgatcgcgtctcctgg

SEQ ID NO: 8 - DNA, 16 bp, Artificial Sequence
  M13F
gtaaaacgacggccag

SEQ ID NO: 9 - DNA, 17 bp, Artificial Sequence
  M13R
caggaaacagctatgac

SEQ ID NO: 10 - protein, 420 aa, Artificial Sequence
  Spike protein (aac 251-670)
PGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGI

SEQ ID NO: 11 - protein, 223 aa, Artificial Sequence
  Spike protein (Full RBD, RBD1)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 12 - protein, 257 aa, Artificial Sequence
  Spike protein (Full RBD, RBD1-L)
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSSFHQLPARSPLPGGGGSFYPSYHSTPQRP

SEQ ID NO: 13 - protein, 211 aa, Artificial Sequence
  Spike protein (RBD0)
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 14 - protein, 245 aa, Artificial Sequence
  Spike protein (RBD0-L fusion protein)
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGGGGSSFHQLPARSPLPGGGGSFYPSYHSTPQRP

SEQ ID NO: 15 - protein, 263 aa, Artificial Sequence
  Spike protein (RBD2)
AVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTG

SEQ ID NO: 16 - protein, 297 aa, Artificial Sequence
  Spike protein (RBD2-L fusion protein)
AVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGGGGGSSFHQLPARSPLPGGGGSFYPSYHSTPQRP

SEQ ID NO: 17 - protein, 418 aa, Artificial Sequence
  NC protein
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 18 - protein, 453 aa, Artificial Sequence
  NC protein, NC-L fusion protein
SDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQAGGGGGSSFHQLPARSPLPGGGGSFYPSYHSTPQRP

SEQ ID NO: 19 - protein, 120 aa, Artificial Sequence
  NC protein (LKR region)
FYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQE

SEQ ID NO: 20 - protein, 155 aa, Artificial Sequence
  NC protein (LKR-L fusion protein)
FYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQEGGGGGSSFHQLPARSPLPGGGGSFYPSYHSTPQRP

SEQ ID NO: 21 - protein, 306 aa, Artificial Sequence [sequence omitted]

SEQ ID NO: 22 - protein, 341 aa, Artificial Sequence
  Mpro protein (Mpro-L fusion protein)
SGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQGGGGGSSFHQLPARSPLPGGGGSFYPSYHSTPQRP

SEQ ID NO: 23 - DNA, 23 bp, Artificial Sequence
  BM1
gttaacagagatttacacagagg

SEQ ID NO: 24 - DNA, 23 bp, Artificial Sequence
  BM2
gcagccctccactcccatggagg

SEQ ID NO: 25 - DNA, 79 bp, Artificial Sequence
  fdx promoter
attactttaaaaattaataaaaacatggtaaaatataaatcgtataaagttgtgtaatttttaaggaggtgtgttacat

SEQ ID NO: 26 - protein, 25 aa, Artificial Sequence
  nprM3
MKSKKLLATVLSAVITLSTVSAVYA

SEQ ID NO: 27 - protein, 35 aa, Artificial Sequence
  L1-2
GGGGGSSFHQLPARSPLPGGGGSFYPSYHSTPQRP

SEQ ID NO: 28 - DNA, 895 bp, Artificial Sequence
  PptbnprM3-RBD1
taagtcagcagaaagtataatgagaaaatataaaatataaataattttctaaaaaacttaacttcatgtgaaaagtttgttaaaatataaatgagcacgttaatcatttaacatagataattaaatagtaaaagggagtgtacgaccaatgaaaagtaaaaaattattagctacagtgctaagtgctgtaatcactctttctactgtttctgcagtttatgctagagttcaaccaacagagtctatagtgagatttcctaatattacaaatctttgcccattcggtgaggtattcaacgctacaaggtttgcaagtgtttacgcttggaataggaaaagaatttctaattgtgttgctgattattcagtattatacaatagtgcttcattctcaacatttaagtgttatggagtaagtccaacaaagttgaacgatttatgtttcacaaatgtatatgctgatagtttcgtgattaggggagatgaagtaagacaaattgcacctggtcagacaggaaaaattgctgactacaattacaagttgcctgatgattttacaggatgtgtaatagcatggaacagtaacaacttagatagtaaggtgggaggaaactataattacttatacagactattcaggaaatcaaacttaaaaccttttgaaagagacatatcaacagaaatataccaggctggtagtacaccatgcaatggtgtggaaggatttaattgttattttcctcttcaatcatacggttttcaacctactaacggtgtaggataccaaccttatagggtagtggtgttgtcatttgagcttttacacgcacctgctactgtgtgtggacctaagaagtctactaatctagtgaagaacaaatgcgtaaacttttaa

SEQ ID NO: 29 - DNA, 826 bp, Artificial Sequence
  PfdxnprM3-RBD1
attactttaaaaattaataaaaacatggtaaaatataaatcgtataaagttgtgtaatttttaaggaggtgtgttacatatgaaaagtaaaaaattattagctacagtgctaagtgctgtaatcactctttctactgtttctgcagtttatgctagagttcaaccaacagagtctatagtgagatttcctaatattacaaatctttgcccattcggtgaggtattcaacgctacaaggtttgcaagtgtttacgcttggaataggaaaagaatttctaattgtgttgctgattattcagtattatacaatagtgcttcattctcaacatttaagtgttatggagtaagtccaacaaagttgaacgatttatgtttcacaaatgtatatgctgatagtttcgtgattaggggagatgaagtaagacaaattgcacctggtcagacaggaaaaattgctgactacaattacaagttgcctgatgattttacaggatgtgtaatagcatggaacagtaacaacttagatagtaaggtgggaggaaactataattacttatacagactattcaggaaatcaaacttaaaaccttttgaaagagacatatcaacagaaatataccaggctggtagtacaccatgcaatggtgtggaaggatttaattgttattttcctcttcaatcatacggttttcaacctactaacggtgtaggataccaaccttatagggtagtggtgttgtcatttgagcttttacacgcacctgctactgtgtgtggacctaagaagtctactaatctagtgaagaacaaatgcgtaaacttttaa

SEQ ID NO: 30 - DNA, 928 bp, Artificial Sequence
  PfdxnprM3-RBD1-L
attactttaaaaattaataaaaacatggtaaaatataaatcgtataaagttgtgtaatttttaaggaggtgtgttacatatgaaaagtaaaaaattattagctacagtgctaagtgctgtaatcactctttctactgtttctgcagtttatgctagagttcaaccaacagagtctatagtgagatttcctaatattacaaatctttgcccattcggtgaggtattcaacgctacaaggtttgcaagtgtttacgcttggaataggaaaagaatttctaattgtgttgctgattattcagtattatacaatagtgcttcattctcaacatttaagtgttatggagtaagtccaacaaagttgaacgatttatgtttcacaaatgtatatgctgatagtttcgtgattaggggagatgaagtaagacaaattgcacctggtcagacaggaaaaattgctgactacaattacaagttgcctgatgattttacaggatgtgtaatagcatggaacagtaacaacttagatagtaaggtgggaggaaactataattacttatacagactattcaggaaatcaaacttaaaaccttttgaaagagacatatcaacagaaatataccaggctggtagtacaccatgcaatggtgtggaaggatttaattgttattttcctcttcaatcatacggttttcaacctactaacggtgtaggataccaaccttatagggtagtggtgttgtcatttgagcttttacacgcacctgctactgtgtgtggacctaagaagtctactaatctagtgaagaacaaatgcgtaaactttggtggaggaggttcatcatttcatcaattaccagctcgttcaccattaccaggaggtggtggatcattttatccatcatatcattcaactccacaacgtccataa

SEQ ID NO: 31 - DNA, 1411 bp, Artificial Sequence
  PfdxnprM3-NC
attactttaaaaattaataaaaacatggtaaaatataaatcgtataaagttgtgtaatttttaaggaggtgtgttacatatgaaaagtaaaaaattattagctacagtgctaagtgctgtaatcactctttctactgtttctgcagtttatgcttctgataatggaccccaaaatcagagaaatgcacccaggattacatttggtggaccctcagattcaactggcagtaaccagaatggagaacgtagtggggctagatcaaaacaacgtaggccccaaggtttacccaataatactgcctcttggttcaccgctcttactcaacatggcaaggaagatcttaaattccctagaggacaaggcgttccaattaacaccaatagcagtccagatgaccaaattggctactaccgtagagctaccagaaggattcgtggtggtgacggtaaaatgaaagatcttagtccaagatggtatttctactacctaggaactgggccagaagctggacttccctatggtgctaacaaagacggcattatatgggttgcaactgagggagccttgaatacaccaaaagatcacattggcaccaggaatcctgctaacaatgctgcaatcgtgctacaacttcctcaaggaacaacattgccaaaaggcttctacgcagaagggagcagaggcggcagtcaagcctcttctcgttcctcatcacgtagtaggaacagttcaagaaattcaactccaggcagcagtaggggaacttctcctgctagaatggctggcaatggcggtgatgctgctcttgctttgctattgcttgacagattgaaccagcttgagagcaaaatgtctggtaaaggccaacaacaacaaggccaaactgtgactaagaaatctgctgctgaggcttctaagaagcctaggcaaaaacgtactgccactaaagcatacaatgtaacacaagctttcggcagacgtggtccagaacaaacccaaggaaattttggggaccaggaactaattagacaaggaactgattacaaacattggcctcaaattgcacaatttgcccccagcgcttcagcattcttcggaatgtctcgtattggcatggaagttacaccttctggaacatggttgacctacacaggtgccattaaattggatgacaaagatccaaatttcaaagatcaagtgattttgcttaataagcatattgacgcatacaaaacattcccaccaacagagcctaaaaaggacaaaaagaagaaggctgatgaaactcaagccttaccacagagacagaagaaacagcaaactgtgactcttcttcctgctgcagatttggatgatttctccaaacaattgcaacaatccatgagcagtgctgactcaactcaggcctaa

SEQ ID NO: 32 - DNA, 1513 bp, Artificial Sequence
  PfdxnprM3-NC-L
attactttaaaaattaataaaaacatggtaaaatataaatcgtataaagttgtgtaatttttaaggaggtgtgttacatatgaaaagtaaaaaattattagctacagtgctaagtgctgtaatcactctttctactgtttctgcagtttatgcttctgataatggaccccaaaatcagagaaatgcacccaggattacatttggtggaccctcagattcaactggcagtaaccagaatggagaacgtagtggggctagatcaaaacaacgtaggccccaaggtttacccaataatactgcctcttggttcaccgctcttactcaacatggcaaggaagatcttaaattccctagaggacaaggcgttccaattaacaccaatagcagtccagatgaccaaattggctactaccgtagagctaccagaaggattcgtggtggtgacggtaaaatgaaagatcttagtccaagatggtatttctactacctaggaactgggccagaagctggacttccctatggtgctaacaaagacggcattatatgggttgcaactgagggagccttgaatacaccaaaagatcacattggcaccaggaatcctgctaacaatgctgcaatcgtgctacaacttcctcaaggaacaacattgccaaaaggcttctacgcagaagggagcagaggcggcagtcaagcctcttctcgttcctcatcacgtagtaggaacagttcaagaaattcaactccaggcagcagtaggggaacttctcctgctagaatggctggcaatggcggtgatgctgctcttgctttgctattgcttgacagattgaaccagcttgagagcaaaatgtctggtaaaggccaacaacaacaaggccaaactgtgactaagaaatctgctgctgaggcttctaagaagcctaggcaaaaacgtactgccactaaagcatacaatgtaacacaagctttcggcagacgtggtccagaacaaacccaaggaaattttggggaccaggaactaattagacaaggaactgattacaaacattggcctcaaattgcacaatttgcccccagcgcttcagcattcttcggaatgtctcgtattggcatggaagttacaccttctggaacatggttgacctacacaggtgccattaaattggatgacaaagatccaaatttcaaagatcaagtgattttgcttaataagcatattgacgcatacaaaacattcccaccaacagagcctaaaaaggacaaaaagaagaaggctgatgaaactcaagccttaccacagagacagaagaaacagcaaactgtgactcttcttcctgctgcagatttggatgatttctccaaacaattgcaacaatccatgagcagtgctgactcaactcaggccggtggaggaggttcatcatttcatcaattaccagctcgttcaccattaccaggaggtggtggatcattttatccatcatatcattcaactccacaacgtccataa

(19) United States
(12) Patent Application Publication
(10) Pub. No.: US 2024/0254167 A1
(43) Pub. Date: Aug. 1, 2024

(54) GENETICALLY MODIFIED CLOSTRIDIUM STRAINS EXPRESSING RECOMBINANT ANTIGENS AND USES THEREOF

(71) Applicant: LIVINGMED BIOTECH S.R.L., Liège (BE)

(72) Inventors: Aleksandra KUBIAK, Liège (BE); Tom BAILEY, Liège (BE); Niall BOLLARD, Liège (BE); Philip HITTMEYER, Liège (BE)

(21) Appl. No.: 18/546,427

(22) PCT Filed: Feb. 15, 2022
(86) PCT No.: PCT/EP2022/053715

§ 371 Date: Aug. 14, 2023

(30) Foreign Application Priority Data
Feb. 15, 2021 (EP) 21157234.2

(51) Int. Cl. C07K 14/005; A61K 39/00; A61K 39/215; A61P 37/04

(52) U.S. Cl. CPC C07K 14/005; A61K 39/215; A61P 37/04; A61K 2039/523; A61K 2039/542; C07K 2319/02; C12N 2770/20022; C12N 2770/20034

(57) ABSTRACT

The present invention relates to genetically modified Clostridium strains that provide improved combinations of functional features useful for several medical applications requiring the preparation and administration of antigens. In particular, clinical grade spores generated from the Clostridium strains described herein can be efficiently prepared, stored, formulated for oral administration, and advantageously used in the prevention and/or treatment of infectious diseases, such as COVID-19.

### CROSS-REFERENCE TO RELATED APPLICATIONS

This application is a national phase entry under 35 U.S.C. § 371 of International Patent Application PCT/EP2022/053715, filed Feb. 15, 2022, published as International Patent Publication WO 2022/171904 on Aug. 18, 2022, which claims the benefit of European Patent Application EP 21157234.2, filed on Feb. 15, 2021, the contents of all are hereby incorporated by reference.

### REFERENCE TO A SEQUENCE LISTING

The present application contains a Sequence Listing, which has been submitted electronically in ASCII format and is hereby incorporated by reference in its entirety. The ASCII copy was created on Feb. 11, 2022, is named “6-Sequence Listing-TXT.txt” and is 43,153 bytes in size.

### FIELD OF THE INVENTION

The present invention relates to novel Clostridium strains and spores, their method of manufacture, and use in clinical applications, including for a use in treating or preventing infectious diseases.

### BACKGROUND OF THE INVENTION

Clostridia are Gram-positive, anaerobic, endospore-forming bacteria that are grouped under the genus Clostridium and comprise approximately 180 species known to produce different types of acids and other chemical compounds through the degradation of sugars, alcohols, amino acids, purines, pyrimidines, and biological polymers. The sporulation process in Clostridia for survival and reproduction purposes commonly follows the exposure of these bacteria to unfavourable environmental conditions, for instance, conditions relating to pH, heating, freezing, radiation, or oxygen levels. The sporulation process is controlled by a complex cascade of specific proteins and other factors acting at the transcriptional or posttranslational level (Shen S et al., 2019).

The replication and other biological properties of Clostridium species have been studied for several purposes. Various technologies relating to culturing, administering, selecting, or otherwise using Clostridia, are known, in particular by modifying a Clostridium genome with regulatory or coding sequences from other organisms that have been modified, cloned, added, disrupted, and/or placed under specific systems for the control of gene expression. As recently reviewed in the scientific literature (McAllister K and Sorg J, 2019; Kwon S et al., 2020), a series of improvements has emerged for engineering and selecting strains from some Clostridium species featuring efficient, permanent, and precise modifications of clostridial genomes, thereby permitting a better understanding of Clostridium biology and improving their utility when provided as cells or as spores. However, mechanisms that exploit the natural homologous recombination pathways present in the Clostridium species have been applied with uneven success and efficacy.

The use of allelic exchange systems, CRISPR-based genetic editing, and other gene expression control systems may allow for the modification of clostridial genomes and then the selection of those clones or variants suitable for a contemplated use. Several approaches have been developed by making use of constructs that provide specific functional, accurate clostridial genome modification(s) (including nucleotide substitution, gene deletion and cassette insertion). Such recombinant Clostridium strains may present specifically modified features relating to sporulation, additional or enhanced enzymatic activity, expression of codon-optimized, non-clostridial genes, expression of a clostridial gene under the control of external inducers, resistance to chemical compounds or conditions, virulence control, or simple cell labelling.

The combination of cloned, recombinant sequences and Clostridium-specific genetic modifications have mostly succeeded in generating single, specifically defective or repressed genes in a laboratory setting. Notably, the use of Clostridium strains in more demanding applications, such as in clinical drug development programs has been much more restrictive, since improved functional properties must necessarily be associated with both strict biocontainment and a tight control of gene expression and replication in vivo. Indeed, Clostridium species are known to specifically accumulate in hypoxic or anoxic environments of the human body, mostly concerning immunological “niches” being either physiological (such as the bone marrow, lymphoid tissue, and intestinal mucosa) or pathological such as those within solid tumours or in chronically inflamed or ischemic tissues (Taylor C and Colgan S, 2017). The clinical use of Clostridium-based therapies has been proposed and tested in several models, in particular in an oncological context for eliciting immunological responses, targeting cancer cells, delivering cytokines, and/or activating prodrugs in Clostridium-infected tumours (Ni G et al., 2019; Torres W et al., 2018; Minton N et al., 2016; WO2009/111177; WO2015/075475; WO2020/157487).

At present, the scientific literature contains few reports regarding how the genome of Clostridium species may be modified in precise and efficient manner to express antigens in human subjects, in particular to provide Clostridium-based preparations that can be used to prevent infection, re-infection, spreading, or pathogenic consequences of viruses, bacteria, and other pathogenic agents. For instance, such literature is typically limited to the use of C. Taeniosporum, C. Difficile, C. Perfringens, or of bacteria including Lactobacilli or Bacillus subtilis, which have immunogenic features that can expose clostridial antigens for vaccination on a cell or spore surface (see e.g., Lin P et al., 2020; Ma S et al., 2018; Wang Y et al., 2018; U.S. Pat. Nos. 10,668,140; 5,800,821; WO2002/046388; WO2011/160026; WO2003/074682; WO2010/006326; WO2005/028654; WO2008/017483; WO2012/001000; WO2014/168497).

It would be advantageous to generate Clostridium-based preparations comprising stable, non-toxigenic, and viable spores that, once administered, preferably orally, can germinate in specific tissues, proliferate, and then express antigens capable of establishing an immunogenic response useful for vaccinating populations against a pathogenic or infectious agent. In addition, such preparations can be useful for establishing other treatment regimens that may include the administration of other drugs targeting the agent or related symptoms caused by the agent, in particular with respect to viral infections. There is currently a need to improve conventional Clostridium-based pharmaceutical formulations for adequately exploiting and controlling their physiological activity, especially when administered in a spore format for the purposes of recombinant antigen expression with enhanced immunogenicity, replication control, achieving a specific localization upon administration, and/or for interactions with specific cells in the human organism providing for efficient administration and the desired immunogenic response.

### SUMMARY OF THE INVENTION

The present invention relates to spore-forming Clostridium strains generated from a combined approach of both targeting and modifying sequences in the Clostridium genome using non-clostridial gene sequences encoding recombinant antigens for eliciting an immunological response in mammalian subjects for the elimination of unwanted cells or pathogens, including bacteria or viruses. Genetically modified Clostridium strains can be generated and advantageously used to prepare cell or spore preparations offering substantial clinical utility associated with the control of Clostridium cell proliferation, both in vivo and in vitro. Such novel strains may be used, alone or in combination with other prophylactic or therapeutic treatments, and can be adapted to specific conditions or infections, in particular those requiring treatment modifications in response to novel pathogens and/or viruses and their novel, more harmful variants.

The approaches disclosed herein further allow establishing improved pharmaceutical compositions and uses involving Clostridium cells and spores for delivering optimized, cell targeting and/or adjuvant-containing recombinant antigens as means for vaccinating subjects, in particular by oral administration. As required by the spreading of virus and bacteria in a subject populations, the genetically modified Clostridium cells or spores can be generated according to the present invention and engineered to express and deliver one or more recombinant antigens in vivo, as identified in a specific organism, strain, or epidemiologically relevant variant, or by combining immunogenic elements from different proteins, strains or organisms. The specific dosage regimens and/or in sub-populations of patients can benefit from optimized, Clostridium-based vaccination protocols involving the administration of a single agent that combines the specific in vivo expression, adjuvant, and/or targeting features of relevant recombinant antigen(s).

In one embodiment of the invention, the novel Clostridium strains present both the expression of a heterologous gene coding for at least one recombinant antigen and also present one or more additional genome modifications for deleting and/or inactivating (or at least attenuating) at least an additional Clostridium gene. In particular, the expression of the Clostridium gene may negatively impact the use of the Clostridium strain for clinical applications, such as encoding an undesired toxic activity (e.g., haemolysis) or metabolic activity (e.g., use of specific nutrients for growth and replication, where the absence of such gene determines auxotrophy, that is, the inability of an organism to synthesize an organic compound required for its growth, replication, or other essential biological activities. Additionally, these Clostridium strains may also present an inducible or repressible sporulation phenotype results from the deletion, substitution, or other genetic modification of the sequence that controls the expression of one or more clostridial genes involved in the sporulation process. The choice of the sporulation gene to be targeted is preferably made from among those genes that are at an early stage of this process, such as at the initiation of the sporulation process up to the establishing of an asymmetric septum (even before the formation of pre-spore), but with limited effect over normal strain replication and metabolism, as described in the literature (Shen S et al., 2019).

These novel Clostridium strains are preferably modified through the introduction and the basal or inducible expression of a heterologous gene expressing a protein containing a non-clostridial, recombinant antigen, within the location of a specifically inactivated Clostridium gene or any other location in the genome of a Clostridium strain. In particular, the heterologous gene may be isolated from preferably viral, or non-clostridial bacterial genome and codes for a protein, modified or unmodified, having antigenic properties associated to any biological or cellular property, including but not limited to human cell- or protein-binding properties, cell de-/differentiating, enzymatic activities, cytotoxic or antiproliferative properties, structural properties, or other observable effect on general human cellular or physiological functions such as signalling, replication (cellular, bacterial, and/or viral), metabolism, aging, pro- or anti-immune response, cytotoxicity, (pro- or anti)necrosis, (pro- or anti)apoptosis, or cell (de)differentiation.

The recombinant antigen is preferably expressed by the novel Clostridium strains as a fusion protein that is secreted in the extracellular space (referred to as “BTA”), or otherwise exposed to circulating immune cells and antibodies, since containing a signal sequence for efficient secretion. A BTA may also comprise protein sequences having adjuvant properties and/or with cell-targeting properties. A genetically modified Clostridium strain may express at least two recombinant antigens from the same or different organism, in a single, two, or more distinct BTAs.

The resulting Clostridium strains disclosed herein can be used for producing purified and/or concentrated cell preparations (or corresponding spore preparations) to be stored or used as pharmaceutical formulations directly in a clinical context, for instance, administered in order to provide a patient in need thereof with the recombinant antigen comprised in the BTA at an effective prophylactic or therapeutic dose. When the pharmaceutical use is associated to additional requirements, including requirements relating to oral administration, the formulation may further comprise supplementary chemical components, such as appropriate additives, salts, stabilizers, adjuvants, or excipients for producing batch or ready-to-use doses, for instance as tablets or capsules, or formulated by mixing BTA-expressing cells (or, preferably, spores) together with food or beverage for subsequent ingestion.

A representation of a general strategy for obtaining the presently disclosed genetically modified Clostridium strains is shown in FIG. 1, which schematically summarizes the main features of the genetically modified Clostridium strains (indicated as CLOSTRAV) and of the related vectors and preparations that are used throughout the instant disclosure. The CLOSTRAV strain belongs preferably any species of the Clostridium genus, including C. (Clostridium) butyricum, C. sporogenes, C. novyi, C. novyi-NT, or C. difficile, or other species known for their properties of clinical interest, which are suitable for the expression of one or more recombinant antigens, the administration in the form of spores, and in vivo proliferation of spore-generated cells in specific locations (such as anoxic areas within the gut or other intestinal tissues) where an immunological response can be triggered for the purpose of vaccination against infectious agents, in particular viruses.

In particular, a novel Clostridium strain that is modified to express a recombinant antigen, is identified herein as CLOSTRAV-BTA, which can be originated through one or more intermediate Clostridium strains that present at least one deleted, inactivated (or at least attenuated) gene, such that the strain lacks toxigenic features when administered in humans or animals. Specific CLOSTRAV and CLOSTRAV-BTA strains are identified by means of two or more modifications in their genomes that are combined to obtain the desired properties. For instance, CLOSTRA-A is a recombinant Clostridium strain in which one, two, or more independent locus or genes are inactivated, or at least attenuated, through the deletion and/or substitution of a genomic sequence, with or without integrating an exogenous marker or functional sequences at this genomic location.

In a preferred embodiment, the genetically modified Clostridium strain expresses at least one recombinant antigen as a BTA and presents one or more additional genome modifications for deleting and/or to inactivating at least one Clostridium gene (or more, in the case of two Clostridium genes), wherein said Clostridium strain is modified to inactivate, delete, or attenuate: a) at least one Clostridium gene encoding a toxic activity; and. b) at least one Clostridium gene encoding a metabolic activity, in particular the ability to synthesize an organic compound required for the growth of said strain.

The genetically modified Clostridium strain preferably presents one or inactivated, deleted, or attenuated genes that are responsible of haemolysis, such as those present in Sag operon. The genetically modified Clostridium strain may further present a defective or attenuated ability to synthesize a precursor for DNA or RNA synthesis (such as uracil) or an essential amino acid (such as tryptophan). The genetically modified Clostridium strain may also present an inducible or repressible sporulation gene selected from Spo0A or SpollAA. The genetically modified Clostridium strain preferably expresses the recombinant antigen as a fusion protein comprising a signal sequence for secretion, a cell targeting sequence and/or an adjuvant sequence, and more preferably the fusion protein comprises a signal sequence for secretion and a cell targeting sequence that targets the recombinant antigen to M cells. The BTA may comprise and raise an immunogenic response against an antigen that is expressed by a virus or a bacterium. In the case of virus, the recombinant antigen may comprise a protein sequence that is present on the surface of the virus, such the receptor binding domain (RBD) that permits entry of the virus into the cell, and consequently, viral infection. As shown in the Examples, a BTA may be designed on the basis of specific sequences within proteins of Coronaviruses, and in particular within the Spike (S) protein or Nucleocapsid (NC) protein sequence of SARS-COV-2 that may also contain mutations characterizing novel variants of medical and biological interest against which an immunogenic response may be useful.

When the BTA is based on a recombinant antigen of SARS-COV-2, the antigen is preferably comprised in the Spike (S), Nucleocapsid (NC), or M^{Pro} protein sequence. The Examples provide details of the DNA sequences that can be constructed and integrated in vectors and CLOSTRAV genomes according to the present invention. Preferred Spike (S) protein sequences and Nucleocapsid (NC) protein sequences herein are those expressed as, e.g., RBD1 protein (SEQ ID NO: 11), RBD1-L fusion protein (SEQ ID NO: 12), RBD0 protein (SEQ ID NO: 13), RBD0-L fusion protein (SEQ ID NO: 14), RBD2 protein (SEQ ID NO: 15), RBD2-L fusion protein (SEQ ID NO: 16), NC protein (SEQ ID NO: 17), NC-L fusion protein (SEQ ID NO: 18), LKR region of NC protein (SEQ ID NO: 19), or LKR-L fusion protein (SEQ ID NO: 20). Alternatively, variants of these protein sequences may be expressed as a BTA having at least 90% identity thereto, including a number of mutations that are found in one or more SARS-COV-2 variants of medical interest, for instance, as identified in FIG. 6B, and FIG. 7B, or in the relevant official webpages and scientific literature as Variant-of-Concern or Variant-of-Interest. Specific constructs and coding DNA sequences for expressing such BTA are disclosed herein, in particular for expressing and secreting Spike (S) or Nucleocapsid (NC) protein sequences, with or without being fused to other protein sequences (PptbnprM3-RBD1, SEQ ID NO: 28; PfdxnprM3-RBD1, SEQ ID NO: 29; PfdxnprM3-RBD1-L, SEQ ID NO: 30; PfdxnprM3-NC, SEQ ID NO: 31; PfdxnprM3-NC-L, SEQ ID NO: 32).

As disclosed herein (e.g., in the Detailed Description and in the Examples), the non-toxigenic CLOSTRA-A strain can be used as a CLOSTRAV strain for generating the CLOSTRAV-BTA strains useful for vaccination, in particular for administering one or more recombinant antigens by means of in vivo proliferating, BTA-secreting cells. Alternative CLOSTRAV strains can be generated and identified through combinations of one, two, or more independent locus or genes that are deleted, inactivated, or at least attenuated. Each of such locus or gene can be potentially useful as a reference “platform” strain to be modified for expressing and delivering in vivo at least one recombinant antigen for a specific pathogenic agent at a desired location by introducing one or more heterologous genes expressing the desired BTA, which can comprise an antigen expressed by a virus or a bacteria (in particular on its surface). In this manner, a series of alternative, numbered, and referred to herein as CLOSTRAV-BTA strains having specific properties and uses may be generated for vaccinating subjects against specific bacteria, viruses (such as a Coronavirus or an influenza virus), plant-derived allergens, and other pathogens or allergens in general, or against specific strains causing more harmful effects in a specific population or areas. Each CLOSTRAV-BTA strain can be stored, characterized, validated and used as such or alternatively as a corresponding spore (each identified as “CBTAS”) and formulations (each identified as “CBTAS-F”).

The novel Clostridium strains described herein can be produced using conventional techniques permitting the modification of a Clostridium genome in a specific and controlled manner, such as by means of plasmids and other constructs wherein the sequences to be integrated in the Clostridium genome are appropriately cloned, modified, oriented, and ordered in the CLOSTRAV genome, using standard recombinant DNA technologies applicable to Clostridium strains. In particular, such Clostridium strains can be modified by using plasmids allowing the integration of sequences in Clostridium genome by exploiting their own homologous recombination system, for example, by using approaches based on CRISPR-Cas9 and other similar gene editing technologies (Liu G et al., 2022).

Exemplary, schematic representations of strategies and vectors for generating the novel Clostridium strains disclosed herein for the clinical uses are provided in FIGS. 2-5. The vectors, generically referred to as pPM-1nn and pPM-2nn, are designed for modifying Clostridium strains and sequentially generating CLOSTRAV and CLOSTRAV-BTA strains that can be characterized and compared in various cell culture conditions, in particular with respect to BTA expression and secretion or general features such as growth conditions or sporulation. These and other features may be further evaluated after in vivo administration, such as immunogenic properties, localization in organisms, effects on specific tissues and organs, effects on metabolism and other physiological functions, or cell proliferation.

The resulting CLOSTRAV and CLOSTRAV-BTA strains should preferably not produce, or least present reduced, undesirable effects otherwise known to be caused by the Clostridium strains in vivo (e.g. haemolysis in human subjects, use of specific molecules as a source of energy or biological building blocks, and/or growth and replication in specific tissues) that are not compatible for clinical uses, such as vaccination. CBTAS and CBTAS-F can be efficiently and safely administered as cells (or spores) within a composition for use as a medicament, optionally comprising an additive, carrier, adjuvant, vehicle, diluent, salts, and/or excipient. Such composition can be provided as a liquid, solid, frozen, dried, and/or lyophilized format, preferably as a formulation suitable for oral administration or injectable within specific tissues.

When intended for use as a vaccine (in vaccination methods and other-vaccine based regimens), the composition may elicit an immunological response in humans or animals (e.g. against a specific viral, plant or bacterial protein, such as one of those exposed on their surface), leading to vaccination or immunization against such virus or bacteria (or, in the case of a plant antigen, tolerance to such antigen). The composition can be also administered in combination with other compounds having one or more prophylactic or therapeutic effects, preferably for vaccination, such as cytokines, interferons, anti-inflammatory compounds, vitamins, inhibitors of bacterial or viral replication, antibodies that block virus entry or activate specific immunological responses, as well as other compounds for treating specific symptoms or pathologies consequent to the infectious disease (or other immunological dysregulation). Additional products and methods can be associated with the production and use of the novel Clostridium strains disclosed herein, in particular when the composition is administered in methods for preventing or treating an infectious disease (for instance those consequent to the infection by a Coronavirus, such as COVID-19), using established clinical regimens (e.g. comprising two or more administrations, in combination with an adjuvant or other preventive treatment).

Novel clostridial cell culture methods are also described herein for providing one or more of cells and spores that are compliant with regulatory and practical requirements including as GMP (Good Manufacturing Practices), clinical procedures, or environmental obligations applicable to genetically modified organisms, with absolute control of sporulation during the manufacturing process and in the event the Clostridium cells are used in vivo.

Additional embodiments according to the present invention relating to specific novel medical products and uses, including the vaccination against specific infective agents (such as SARS-COV-2, using the BTA cassette arrangements and recombinant antigens disclosed in FIGS. 6-8), are described herein and in the Examples below.

### BRIEF DESCRIPTION OF THE DRAWINGS

FIG. 1: Overview of an exemplary process for generating Clostridium strains that produce spores to be used prophylactically or therapeutically, such as in vaccination protocols, which are obtained from a CLOSTRAV-BTA strain and result from the sequential modification of a Clostridium strain with one or more pPM-1nn and pPM-2nn vectors and with the preparation and optimization of final formulations at the level of the storage conditions and/or composition format. The instant pPM-1nn vector is useful to modify, add, and/or delete one or more genetic element(s) that control features relevant for medical uses or safety. The instant pPM-2nn vector comprises and provides for expressing a sequence coding for a biological antigen sequence that is fused with one or more sequences (e.g., coding for a signal sequence, an adjuvant, and/or a cell targeting sequence) in a single fusion protein referred to as BTA. CLOSTRAV-BTA strains are cultured, selected and optimized for in vitro production prior to in vivo administration of the corresponding spores (CBTAS) and spore formulation (CBTAS-F) that generate Clostridium cells containing, secreting or exposing a BTA, e.g., a recombinant antigen that can act as an immunogen against an infectious agent, fused or not with a sequence permitting its secretion and/or targeting to specific cells.

FIG. 2: Schematic representation of vectors and of the biological mechanisms for generating CLOSTRAV and CLOSTRAV-BTA. Both of the pPM-1nn (FIG. 2A) and pPM-2nn (FIG. 2B) vectors are formed by a backbone (comprising elements allowing the selection, maintenance and replication in Gram-negative and Gram-positive bacteria, the latter can be made possible by the TraJ gene that is required for a correct replication and conjugation in the Clostridium species), and a Clostra Cassette (including the elements necessary for modifying a clostridial genome). The Clostra Cassette includes three primary elements (F1, F2, and F3) for directing homologous recombination within the CLOSTRA genome using a CRISPR-Cas9 approach. A first element includes a Cas9 gene to be expressed into CLOSTRA (F1). A second element includes a sgRNA module designed to specifically guide the CRISPR/Cas9 modification into a CLOSTRA or CLOSTRAV genome (F2). A third element (F3) comprises a Left Homology Arm and a Right Homology Arm (referred to as LHA and RHA, respectively) which flank the sequence to be introduced in clostridial genome. This element mainly differentiates pPM-1nn from pPM-2nn vector, since the former includes a heterologous sequence (HetSeqⁿ, referring to coding, regulatory, tag sequences, or incomplete or complete genes that is used to obtain the desired CLOSTRAV genome and phenotype) and the latter contains all the genetic elements that allow the correct expression and secretion of a recombinant antigen, in particular a sequence coding a biological antigen sequence, fused (or not) with sequences encoding a biological adjuvant and/or targeting sequences in a single coding sequence (BTA cassette; BTA^{c}). All of these elements can be sourced from commercially available plasmids or alternatively, amplified from genomic, transcribed, or retro-transcribed nucleic acid having any other origin. These elements can be cloned either within the plasmid backbone or in the Clostra Cassette in the order shown, or any other order that provides the desired expression and/or integration of the cloned nucleic acids (also in view of the disclosure of PCT/EP2020/087338). The size and orientation of genetic elements as shown is not representative for all plasmids that may be derived from pPM-1nn and pPM-2nn reference constructs. FIG. 2C: a clostridial genome can be modified at a given position (Locusⁿ) by CRISPR/Cas9-guided homologous recombination using pPM-1nn vectors, here represented schematically, by showing only the Clostra Cassette relevant for modifying the CLOSTRA genome with the HetSeqⁿ sequence that is cloned in F3 of Clostra Cassette of a pPM-1nn vector (this mechanism similarly applies to pPM-2nn vectors as well). Following a double-stranded break of the CLOSTRA genome by Cas9 (due to the activity of coding and regulatory sequences in F1 and F2 of Clostra Cassette), cells that repair the break by the high-fidelity, homology-directed repair pathway, using pPM-1nn plasmid as a template for repair, will remove the gRNA target site and introduce HetSeqⁿ, thereby re-establishing the integrity of Clostridium genome and allowing survival of only of those cells comprising such CLOSTRA-derived genome. This approach can be then re-applied to such CLOSTRAV genome using one or more pPM-2nn vectors for obtaining CLOSTRAV-BTA strains that combine safer, biological features with the functional elements for expressing and secreting a recombinant antigen as a BTA by means of cells germinated from CBTAS.

FIG. 3: Schematic representation of exemplary types and combinations of sequences that can be cloned into pPM-1nn or pPM-2nn vectors to be targeted in a Locusⁿ within clostridial genomes and exploited for separately or sequentially “knocking-in” or “knocking-out” full genes, coding sequences, and/or regulatory sequences. FIG. 3A: An exemplary CLOSTRA-derived genome can be modified using sequentially one or more pPM-1nn vectors in which the HetSeqⁿ of Clostra Cassette (being a marker, a coding and/or regulatory sequence) is targeted to a Locusⁿ of interest, for example, a gene for a metabolic activity (Met), a toxic activity (Tox), a structural protein (Str), a replication-regulating factor such as sporulation gene (Spo), any other coding or non-coding gene element (Gen), including any intergenic sequence (Int). The resulting, generic CLOSTRA-Aⁿ strain can be used as CLOSTRAV reference strain. FIG. 3B: an exemplary pPM-2nn vector (pPM-2nn) contains a BTA cassette (BTA^{c}) including a cloning site for a BTA coding sequence that is surrounded by regulatory sequences for transcription (Reg₁ and Reg₂) and terminator sequences (Ter₁ and Ter₂). This arrangement makes BTA^{c} an autonomously transcribed cistron or gene within such vector and, once integrated by CRISPR/Cas9 technology using left- and right homology arms (LHA and RHA) within CLOSTRAV genome, in CLOSTRAV-BTA genome. FIG. 3C: different, exemplary BTA can be expressed in a pPM-2nn vector (or in any other shuttle, intermediate vector) by cloning appropriate coding sequences that form the complete BTA coding sequence. The basic arrangement (BTAc0) includes the coding sequence for the recombinant antigen (Antigen) fused to a sequence that allows the secretion of the resulting fusion protein by CLOSTRAV-BTA strain (Sec). Such arrangement of BTA coding sequences may also include (as shown in exemplary BTAc1-BTAc5 constructs) a sequence coding for a protein that targets the fusion protein to specific cells (Targeting) and/or a sequence coding for a protein having adjuvant activity (Adjuvant) separated by a linker sequence (typically formed by Glycine- and/or Serine-only residues; Linker) in alternative orders and combinations. Moreover, two (or more) distinct antigens with related Adjuvant and/or Targeting sequences can be either co-transcribed in a single protein sequence (as shown in exemplary BTAc6 and BTAc7 constructs) or by forming a bicistronic structure wherein distinct terminator and regulatory, promoter sequences (TRP) are positioned within the BTA cassette (as shown in exemplary BTAc8 and BTAc9 constructs). Sequences coding for additional protein elements (such as a tag, a multimerization domain, or a proteolytic site) can be introduced in a BTA coding sequence by adding the corresponding coding sequence in any of the arrangements described above.

FIG. 4: Designing exemplary pPM-1nn vectors to be used for generating intermediate CLOSTRA-Aⁿ and final CLOSTRAV strains based on Clostridium species such as C. sporogenes (chosen as a representative Clostridium species). FIG. 4A: The generic reference plasmid pPME-101 (also disclosed in PCT/EP2020/087338) is based on the pPM-1nn structure contains a first BookMark sequence (BM1; SEQ ID NO: 23) within the Clostra Cassette (represented here) that can be inserted in the Streptolysin S (“Sag”) operon (from sagA to sagJ), and subsequently generating CLOSTRA-A1 strains with a haemolysin-negative phenotype. Left- and right homology arms (LHA_{sag} and RHA_{sag}) flanking the BM1 sequence are selected and assembled in the F3 element of a Clostra Cassette, so that F1 and F2 elements in Clostra Cassette allow performing CRISPR-Cas9 technology by triggering homologous recombination between the sequences surrounding Sag operon in Clostridium genome and the pPME-101 plasmid. The resulting CLOSTRA-A1 genome and strain can be validated as being non-haemolytic as described in PCT/EP2020/087338. FIG. 4B: The CLOSTRA-A1 strain can be modified with a pPM-1nn vector designated as pPME-105 that contains a BookMark 2 sequence (BM2; SEQ ID NO: 24). Left- and right homology arms (LHA_{pyr} and RHA_{pyr}) flanking the BM2 sequence are selected to trigger homologous recombination between the sequences surrounding the pyrE gene in the CLOSTRA-A1 genome. The deletion of approximately 600 base pairs in pyrE gene from the CLOSTRA-A1 genome allows obtaining a CLOSTRA-A2 strain containing a BM2 tag that is also an uracil auxotroph mutant (failing to grow in media lacking uracil supplementation, as described in PCT/EP2020/087338), and thus suitable as “platform” CLOSTRAV strain. FIG. 4C: Alternative CLOSTRAV-BTA strains including any of the BTA cassettes summarized in FIG. 3C can be introduced in a potential CLOSTRAV (such as CLOSTRA-A1 and CLOSTRA-A2 strains) by choosing the appropriate right/left homology arms and sgRNA to be cloned in pPME-200, an exemplary pPM-2nn vector (only Clostra Cassette is shown). Alternative vectors designated as pPME-210, pPME-220, pPME-230, and pPME-240 allow targeting BTA^{c} to a specific Locusⁿ such as BM1, BM2, pyrE, and Sag operon (respectively), substituting such sequence in the specific CLOSTRAV strain with the BTA coding sequence and obtaining the corresponding CLOSTRAV-BTA strain that expresses the BTA in different locations of a CLOSTRAV genome.

FIG. 5: Schematic representation of two approaches for generating and using exemplary, alternative CLOSTRA-BTA strains disclosed herein based on the same CLOSTRA-A2 strain as the initial CLOSTRAV strain, whose genome is modified using sequentially pPME-101 and pPME-105 vectors to make such strain non-haemolytic and uracil auxotrophic. Either the BM1 (using a pPME-210 vector) or the BM2 (using a pPME-220 vector) tag sequence can be used to introduce a BTA cassette and generating two alternative CLOSTRAV (named CLOSTRAV-A2C1 and CLOSTRA-A2C2), both still non-haemolytic and uracil auxotrophic. These strains can be expanded in cell culture conditions until sporulation is induced (by applying starvation, chemicals, temperature, or other condition), generating the corresponding CBTAS (CLOSTRAV-A2C1S and CLOSTRA-A2C2S) and CBTAS-F (CLOSTRAV-A2C1F and CLOSTRA-A2C2F) having functional, safety, and immunogenic properties that can be tested in animal models prior to administration.

FIG. 6: Designing new CLOSTRAV-BTA strains based on the CLOSTRA-A2 strain (non-haemolytic and pyrE-negative, uracil auxotrophic) that allow generating spore suitable for vaccination against SARS-COV-2 using recombinant variants of RBD domain of Spike protein as immunogen. FIG. 6A: Three types of BTA coding sequences can be designed on the basis of the arrangement of sequences that are shown in FIG. 3C (as BTAc0, BTAc1, and BTAc3) for expressing the receptor binding sequence of Spike (S) protein from SARS-COV-2 virus (RBD) as recombinant antigen (thus referred as BTA-RBDc0, BTA-RBDc1, and BTA-RBDc2). Specific signal sequences (Sec, a specific signal sequence is identified as nprM3; SEQ ID NO: 26), linker sequences (Linker) and cell targeting sequences (L1-2, where two cell targeting sequences may be assembled in a single peptide separated by a Gly/Ser linker, boxed; SEQ ID NO: 27) are assembled with or without a bacterial sequence known to have adjuvant activity (Flagellin C, FliC, from S. typhimurium or E. coli). These BTA coding sequences can be cloned into one of the pPME-200 vectors described in FIG. 4C (pPME-210, pPME-220, pPME-230, and pPME-240) to be subsequently used for generating the corresponding RBD-expressing CLOSTRAV-BTA strains to be tested and compared using the resulting cells or spores in appropriate in vitro and in vivo preclinical assays. FIG. 6B: Sequence of the central portion of Spike (S) protein from SARS-COV-2 (Uniprot accession number P0DTC2, fragment 251-670; SEQ ID NO: 10; corresponding residue number is shown; the protein sequence that is commonly identified as RBD is underlined; SEQ ID NO: 11). The positions that are found most commonly mutated in SARS-CoV-2 biological samples from infected subjects are identified by \hat{ }. Among such positions, those found mutated in one or more of SARS-COV-2 variant-of-concern (according to the official WHO definition and label that is provided at https://www.who.int/en/activities/tracking-SARS-COV-2-variants) are identified by ‡. The partially overlapping, protein sequences presently disclosed as being SARS-COV-2 antigens for producing antibodies and vaccines under development are indicated as RBD0 (SEQ ID NO: 13; the corresponding sequence, when expressed as a BTA-RBDc1 mature protein sequence, is RBD0-L; SEQ ID NO: 14), RBD1 (SEQ ID NO: 11; the corresponding sequence, when expressed as a BTA-RBDc1 mature protein sequence, is RBD1-L; SEQ ID NO: 12), and RBD2 (SEQ ID NO: 15; the corresponding sequence, when expressed as a BTA-RBDc1 mature protein sequence, is RBD2-L; SEQ ID NO: 16). Alternative RBD sequences may be expressed in CLOSTRAV-BTA strains for producing antibodies or vaccines suitable for administration in populations having specific medical needs and/or more directly exposed to different SARS-COV-2 strains and variants, including one or more of the positions indicated above by \hat{ } or ‡ being mutated as defined in the literature for a VOC or a VOI.

FIG. 7: Designing new CLOSTRAV-BTA strains can generate spores suitable for vaccination against SARS-COV-2 using recombinant antigens based on a protein sequence of Nucleocapsid (NC) or Main Protease (Chain C, 3C-like proteinase nsp5; M^{Pro}) protein as the immunogen. FIG. 7A: The construct described in FIG. 6A for expressing the RBD domain as BTA can be adapted to similarly express recombinant variants of NC or M^{Pro} protein as BTA using the BTAc1 cassette structure (thus indicated as BTA-NCc1 and BTA-M^{pro}c1, respectively; other codes and symbols are identical to those shown in FIG. 6A). FIG. 7B: NC protein from SARS-COV-2 (Uniprot accession number P0DTC9, amino acids 2-419; SEQ ID NO: 17; the corresponding sequence, when expressed as a BTA-NCc1 mature protein sequence, is NC-L; SEQ ID NO: 18) contains the linkage region (LKR) in the central portion (fragment 171-290, residue number is shown, SEQ ID NO: 19; the corresponding sequence, when expressed as a BTA-NCc1 mature protein sequence, is LKR-L; SEQ ID NO: 20) that contains most of the positions in NC protein found to be mutated in biological samples from SARS-COV-2 infected subjects and potentially relevant for SARS-COV-2 infectivity and/or activities (identified by \hat{ }). FIG. 7C: Full sequence of M^{pro} protein from SARS-COV-2 (Uniprot accession number P0DTD1 for full Replicase polyprotein 1ab; M^{pro} protein sequence corresponds to residue numbers 3264-3569 in the polyprotein prior to proteolytic maturation; SEQ ID NO: 21; the corresponding sequence, when expressed as a BTA-M^{pro}c1 mature protein sequence, is M^{pro}-L; SEQ ID NO: 22) comprising some positions that are found mutated in biological samples from SARS-COV-2 infected subjects and potentially relevant for SARS-COV-2 infectivity and/or activities (identified by \hat{ }). Recombinant variants of NC and M^{pro} protein sequences (including those presenting one or more of the positions indicated above by \hat{ } being mutated as defined in the literature for a VOC or a VOI of SARS-CoV-2) can be expressed in CLOSTRAV-BTA strains for producing antibodies or vaccines, alone or in combination with each other or with an RBD-containing BTA (such as one of those shown in FIG. 6B), using any of the BTAc6-BTAc9 cassette structures shown in FIG. 3C for assembling such sequences.

FIG. 8: Construction and sequence of a BTA cassette prior of integrating such DNA element in a pPME-200 vector and generating corresponding CLOSTRAV-BTA strains. FIG. 8A: Schematic illustration of pATB1C-41.1 expression vector suitable for transfer by conjugation to CLOSTRA-A2 strain. The exemplary RBD coding sequence (RBD1; see FIG. 6B) has been optimized for expression and secretion in E. coli and Clostridium strains and was inserted into pATB1C vector via type IIs restriction sites (allowing Golden gate DNA assembly) such that it is transcribed under a ptb promoter (Pptb, a promoter adapted from the gene for phosphotransbutyrylase in C. acetobutylicum, ATCC 824; Tummala S et al., 1999), and translated as a fusion protein with a signal sequence (nprM3, associated with the protein coding gene CLSPO_c14710 in C. sporogenes NCIMB 10696). The position of terminators T1 and T2 and of sequences for M13R (standard M13-reverse primer) and M13F (standard M13-forward primer) is indicated together with selection marker (chloramphenicol resistance) and replication elements for Gram-Negative (−) and Gram-positive (+) bacteria. The DNA sequence comprising a Pptb promoter and the sequence coding the nprM3 signal sequence as fused is referred to herein as PptbnprM3-RBD1 (SEQ ID NO: 28). The corresponding protein sequence is expressed as nprM3-RBD1 (later processed as RBD1; SEQ ID NO: 11). A first alternative construct can be generated by putting the BTA coding sequence (nprM3-RBD1, in this case) under the control of the promoter of C. sporogenes ferredoxin gene (i.e., the fdx promoter or Pfdx, associated with the protein coding gene Clspo_c0087; SEQ ID NO: 25) and using RBD1 only (the corresponding DNA is named PfdxnprM3-RBD1; SEQ ID NO: 29) and is cloned in pATB1C-42.1 expression vector (FIG. 8B). The nprM3-RBD1 protein can be also expressed under the Pfdx promoter but linked to cell targeting sequences (shown in FIG. 6A; the corresponding DNA is termed PfdxnprM3-RBD1-L; SEQ ID NO: 30) and is cloned in a pATB1C-42.2 expression vector (FIG. 8C) to express the corresponding nprM3-RBD1-L protein sequence (later processed as RBD1-L; SEQ ID NO: 12). Similarly, a NC protein fragment (SEQ ID NO: 17) can be expressed still under Pfdx promoter as a fusion protein (the corresponding DNA is termed PfdxnprM3-NC; SEQ ID NO: 31) and is cloned in pATB1C-42.3 expression vector (FIG. 8D) to express the corresponding nprM3-NC protein sequence (later processed as NC-L; SEQ ID NO: 17), or cloned together with the cell targeting sequences (as indicated in FIG. 7A; the corresponding DNA is termed PfdxnprM3-NC-L; SEQ ID NO: 32) and is cloned in a pATB1C-42.4 expression vector (FIG. 8E) to express the corresponding nprM3-NC-L protein sequence (later processed as NC-L; SEQ ID NO: 18).

FIG. 9: Validation of constructs comprising a BTA cassette prior of integrating such DNA element in a pPME-200 vector and generating corresponding CLOSTRAV-BTA strains. FIG. 9A: Colony PCR screen in E. coli cells transformed with pATB1C-41.1 expression vector. PCR reaction contained two screening primers (M13F and M13R), the E. coli colony DNA (as template), and DreamTag PCR master mix. The reaction was carried out in accordance with manufacturer's instructions. Colonies 2-8 showing the correct band (approximately 1 kb) are indicated with a star. The sequence of three plasmids (2,3 and 4) extracted from E. coli have been verified by Sanger sequencing, transformed into S17.1 E. coli conjugative donor strain and transferred to CLOSTRA-A2 following the conjugation procedure as described in Materials and Methods. M-DNA molecular marker (NEB, 1 kb plus) is also shown. FIG. 9B: Colony PCR screening of correct pATB1C-41.1 plasmid conjugated to CLOSTRA-A2 strain as CLOSTRAV. PCR reaction contained two screening primers (M13F and M13R), the DNA extracted from each Clostridium colony (as template), and DreamTag PCR master mix. The reaction was carried out in accordance with manufacturer's recommendations. Three CLOSTRA-A2 strains (C1-3; indicated with a star) carrying the PCR-confirmed pATB1C-41.1 plasmid were taken forward to the next stage of investigation (that is Western blotting) and, in parallel, the PCR products were sent for sequencing (C1 actually presented a mutation, and thus was considered for further experiments as control only). M-DNA molecular marker (NEB, 1 kb plus) is also shown (arrow indicates the 1 Kb position).

FIG. 10: Validating the main features of exemplary, recombinant CLOSTRAV-RBDc0 spores and cells expressing a BTA-RBDc0 cassette cloned in pATBC-41.1 vector. FIG. 10A: Spores obtained from a clone identified in FIG. 7C as having integrated the BTA cassette correctly and without sequence mutations (Clone 2, C2), have been used for a preliminary in vitro CBTAS validation by light micrograph. Pictures of RBD-expressing CLOSTRAV-BTA-RBD (Clone 2) were taken at 5-day cultures before (left) and after (right) purification. Vegetative CLOSTRAV-BTA-RBD cells (phase dark rods) are visible in the culture prior to purification, but only CBTAS-RBD (phase bright spheres) is visible following purification. FIG. 10B: CBTAS-RBDC2 (CBTAS-RBD clone 2) stability has been evaluated using colony forming units/ml (CFU/ml) to determine the baseline count prior to storage on day 1 and then compared in equal volumes of CBTAS-RBDC2 preparations that were placed in different storage condition (−20° C., 4° C., room temperature/RT, and 37° C.) in triplicates and tested after 60 days as described in Materials and Methods section. FIG. 10C: cells and corresponding cell culture supernatants of mid-exponential cultures of CLOSTRAV-BTA-RBD (Clones 1-3) expressing plasmid-based SARS-COV-2 antigen were separated by centrifugation. Whole cell and supernatant proteins were heat-denatured in reducing conditions and separated by SDS-PAGE for Western blot. The presence of recombinant RBD antigen was determined using a specific monoclonal antibody (SARS-CoV-2 Spike RBD Mab, Clone 1034515; Cat. No. MAB105401-100) and HRP-conjugated secondary antibody (Rabbit Anti-Mouse IgG H&L, HRP; cat. no. ab6728, Abcam). rRBD; recombinant RBD antigen (Raybiotech, cat. no. 230-01102-100), positive control; WT: wild-type CLOSTRA-A2; P: CLOSTRAV clone transformed with an empty pATB1C vector, not including RBD antigen; C1: CLOSTRAV-BTA-RBDC1 clone 1 transformed with pATB1C-41.1 with mutated DNA sequence for RBD; C2-C3 CLOSTRAV-BTA-RBD clone 2 and clone 3, transformed with pATB1C-41.1 that comprise the correct BTA-RBD coding sequence; M: Protein marker (Precision Plus Protein™ All Blue, Bio-Rad). The lack of detectable products in cell lysates samples indicates complete RBD secretion in culturing media. According to manufacturer's data, the size of rRBD control protein (expressed in E. coli) is approximately 25 kDa. Based on Western blotting results, the size of RBD antigen secreted from recombinant clostridial strains is also evaluated at approximately 25 kDa (see arrow). The visual shift in molecular weight can be explained by upward curving of the protein band at the ends of the gel due to non-optimal parameters of protein gel electrophoresis. These data confirm that Clostra Cassette in a pATB1C-41.1 expression vector can be adapted for generating pPME-200 vectors, corresponding CLOSTRAV-BTA-RBD strains (by CRISPR-Cas9 technology), in addition to related CLOSTRAV-Derived Products (cells, spores, and formulations for medical uses, in particular for vaccination).

FIG. 11: Validation of constructs comprising a BTA cassette under the control of a Pfdx promoter prior to integrating such DNA element in a pPME-200 vector and generating corresponding CLOSTRAV-BTA strains. FIG. 11A: The reaction for a colony PCR screen in CLOSTRA-A1 cells transformed with pATB1C-42.1 plasmid was performed as shown in FIG. 9A for the pATB1C-41.1 plasmid, using the M-DNA molecular marker (NEB, 1 kb plus) for confirming the correct amplification of a 1052 bp fragment in seven clones (identified by a star). FIG. 11B: The reaction for a colony PCR screen in CLOSTRA-A1 cells transformed with a pATB1C-42.2 plasmid was performed as shown in FIG. 9A for pATB1C-41.1, using the M-DNA molecular marker (NEB, 1 kb plus) for confirming the correct amplification of a 1276 bp fragment in two clones (identified by a star). FIG. 11C: The reaction for a colony PCR screen in CLOSTRA-A1 cells transformed with pATB1C-42.4 plasmid was performed as shown in FIG. 9A for pATB1C-41.1, using the M-DNA molecular marker (NEB, 1 kb plus) for confirming the correct amplification of a 1739 bp fragment in three clones (identified by a star). Lane P show an amplification product obtained using a pATB1C-42.4 plasmid (positive control). The correct cloning of the sequences has been confirmed by similarly screening and selecting clones for the pATB1C-42.3 plasmid.

FIG. 12: Design of a study for evaluating whether a protective immune response against SARS-COV-2 infection can be induced in golden hamsters by the oral administration of spores from different transconjugant CLOSTRA-BTA strains that express RBD- or NC-based recombinant antigens by secretion of such antigens in the intestines of the treated animals. Six treatment groups (each including 10 golden hamsters) are established: Treatment with saline solution only (PBS, 250 μl; Group 1); Spores from a CLOSTRA-A1 strain not expressing a BTA (empty pATB1C plasmid; Group 2); Spores from a CLOSTRA-A1 strain expressing RBD antigen with or without a cell targeting sequence (pATB1C-42.1 of FIG. 8B, Group 3; pATB1C-42.2 of FIG. 8C, Group 4); Spores from a CLOSTRA-A1 strain expressing NC antigen with or without a cell targeting sequence (pATB1C-42.3 of FIG. 8D, Group 5; pATB1C-42.4 of FIG. 8E, Group 6). In Groups 2-6, spores are orally administered by gavage (1×10⁸ CFUs in PBS, 250 μl) on day 1 and day 14 (booster). All animals are monitored for signs of distress, infection, changes in body weight, with periodical throat swabs, prior to or following a challenge with SARS-COV2 virus (day 28), faeces is collected (days: 2,3, 15 and 16) and blood is sampled (day 11 and/or 25) for comparative analysis at the end of experiment (day 36 or later, up to day 52). Analysis of the production of anti-S protein (RBD) or NC protein immunoglobulins (IgGs and neutralizing antibodies) is performed in each Group, in parallel to the analysis of faeces, blood, the digestive system and other organs (liver, spleen, kidneys) in sacrificed animals for comparing viral charge and other physiological effects.

### DETAILED DESCRIPTION OF THE INVENTION

### (i) General Definitions

“CLOSTRA” refers to any species, strain, isolate, variant, and cells identified as belonging to the Clostridium genus that present sporulation and germination control in organisms, for example, being directly isolated from organisms and biological samples, or obtained from public sources, including repositories and cell banks. A non-exhaustive list of such specific Clostridium species includes C. (Clostridium) butyricum, C. sporogenes, C. novyi, C. difficile, C. perfringens, C. botulinum, and any isolate or strain found in repositories and cell banks, described in the literature, and/or commonly used in industrial or clinical applications. Additional details and classifications pertaining to Clostridium species to be used as CLOSTRA can be found in the scientific literature (Cruz-Morales P et al., 2019). Moreover, a CLOSTRA strain can be any of recombinant, laboratory Clostridium strains that is indicated as CLOSTRA, CLOSTRA-A, CLOSTRA-S, CLOSTRA-T, and strains derived from them that are described in PCT/EP2020/087338.

Suitable CLOSTRA strains can be identified from those available in public collections such as American Type Culture Collection (ATCC), National Collections of Industrial, Food and Marine Bacteria (NCIMB), National Collection of Type Cultures (NCTC), or from any depositary Institution that is designed as an International Depositary Authority under the Budapest Treaty. Specific listings are available searchable in KEGG website (https://www.genome.jp/kegg-bin/show_brite?br08601.keg) or in NCBI website (e.g., searchable at https://www.ncbi.nlm.nih.gov/genome/browse/#!/prokaryotes/clostridium or in the NCBI Taxonomy Browser under the accession number txid1485 (https://www.ncbi.nlm.nih.gov/Taxonomy/Browser/wwwtax.cgi?mode=Info&id=1485).

The choice of Clostridium species to be used as a CLOSTRA can depend on their biological features and later use of CLOSTRA-Derived Products (including specific CLOSTRAV-BTA strains). For instance, Clostridium species known to colonize human tissues (such as internal organs or skin) are preferred for generating CLOSTRAV for medical uses (e.g., C. butyricum or C. sporogenes), since they are non-toxigenic, or after selecting strains that lack toxic features (as in C. novyi-NT and other non-toxigenic variants of C. botulinumor C. perfringens). In general, the Clostridium species to be used as a CLOSTRA according to the present invention are those species that can integrate into the gut microbiome, in particular the sporobiota where spores can germinate and proliferate (Egan M et al., 2021). A CLOSTRA genome may already present heterologous (non-clostridial) sequences or genomic deletions, rearrangements, mutations, duplications, or other modifications from a reference genome sequence of a Clostridium species unrelated to the use of sequences present in the Clostra Cassette within in a vector that is designed, produced, and used according to the present invention.

“Locusⁿ” refers to any DNA sequence comprised in a CLOSTRA genome suitable for modification according to the methods of the present invention. The DNA sequence into the CLOSTRA genome can be of any size, such as one or more full operons, one or more full genes, a replication site, or intergenic non-coding sequence, as well specific elements within such sequences including entire or partial coding sequences, promoters, or other sequence regulating gene expression or replication of any length and composition.

“HetSeqⁿ” refers to any DNA sequence not comprised in a CLOSTRA genome, which is intended to be non-randomly integrated in a CLOSTRA genome according to the methods of the invention. This DNA sequence can be of any size and origin (including from the genome of a different CLOSTRA, bacteria, yeast, plant, mammal, human, or any man-made variant and artificial sequence) and may include as one or more full operons, one or more full genes, a replication site, or intergenic non-coding sequence, as well specific elements within them such as entire or partial coding sequences, tag sequences, DNA sequences that can be transcribed in specific RNA species, promoters, markers, or other sequence regulating gene expression or replication of any length and composition.

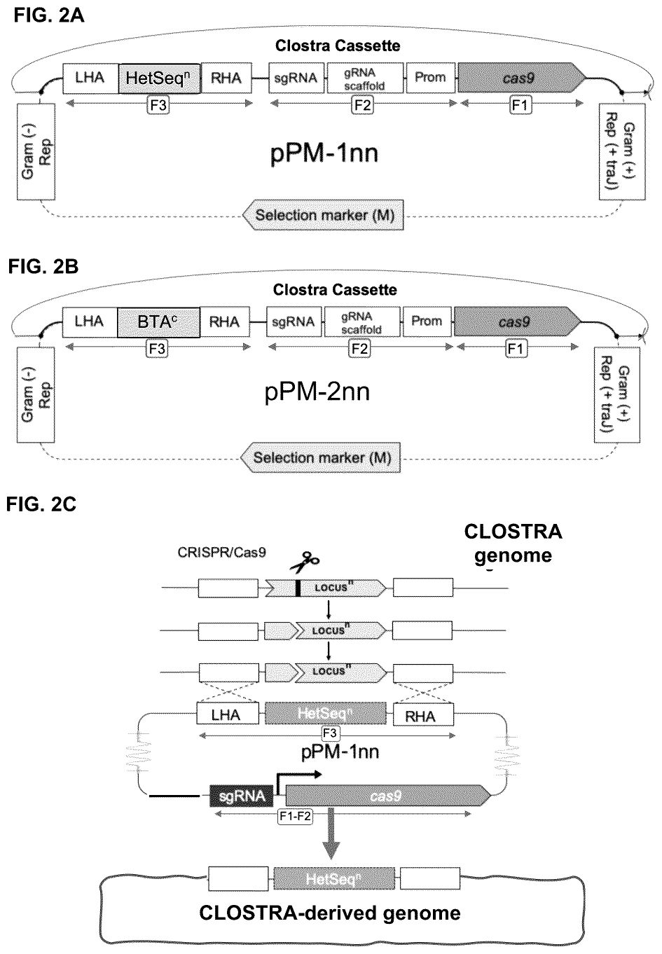

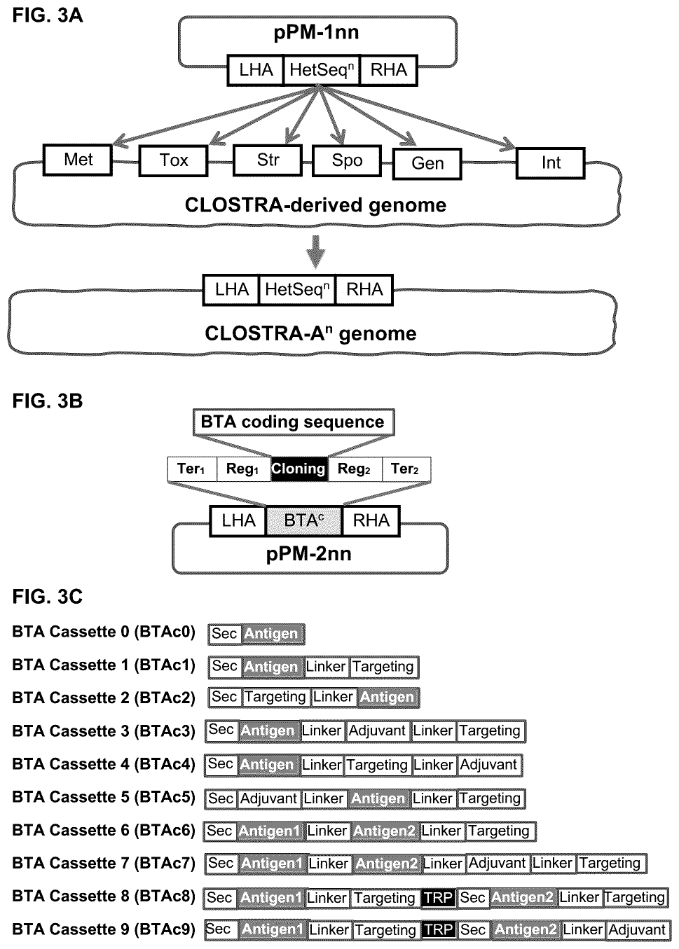

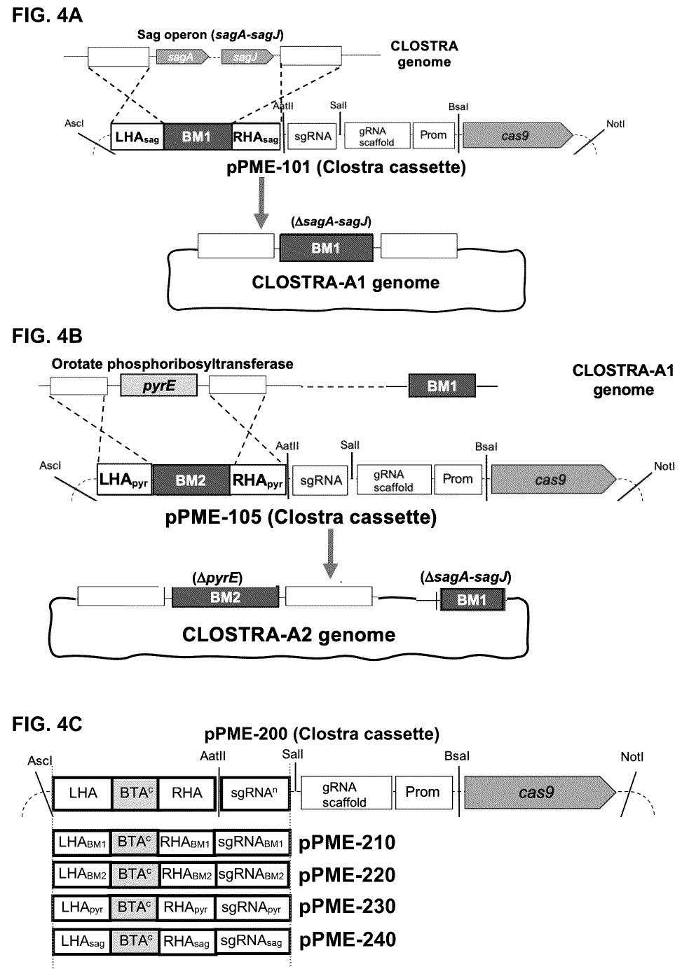

The term “Clostra Cassette” as used herein refers to a recombinant DNA sequence that is cloned in a plasmid or other vector comprising at least a first HetSeqⁿ sequence that can be integrated into a Locusⁿ of a CLOSTRA genome and, preferably, at least a second HetSeqⁿ sequence that allows the stable integration of the first HetSeqⁿ into a Locusⁿ of CLOSTRA genome. The first HetSeqⁿ sequence may contain additional sequences at 5′ and/or 3′ end that are identical or at least highly homologous to the sequence within or surrounding the desired Locusⁿ, thereby triggering a precise integration of the first HetSeqⁿ sequence into the CLOSTRA genome by homologous recombination (also defined as F3). The second HetSeqⁿ sequence may include one or more genes that encode proteins which can facilitate and/or guide the precise integration of DNA sequence in a CLOSTRA genome, as shown in FIG. 2 and FIG. 3 for generic pPM-1nn and pPM-2nn plasmids, and for the pPME-100 and pPME-200 derivative plasmids as shown in FIG. 4, which depicts elements suitable for the application of CRISPR/Cas9 technology within the F1/F2 elements. A Clostra Cassette may be constructed and used in any compatible plasmid or vector for DNA recombinant technologies, but preferably in a vector that can be maintained in both Gram-positive and Gram-Negative bacteria.

The definitions above summarize certain aspects that are generally common to those disclosed by PCT/EP2020/087338 (unless indicated otherwise). Further, specific definitions are provided in the present disclosure with respect to BTA-related embodiments (below, in the Summary of the Invention or in the Description of the Figures).

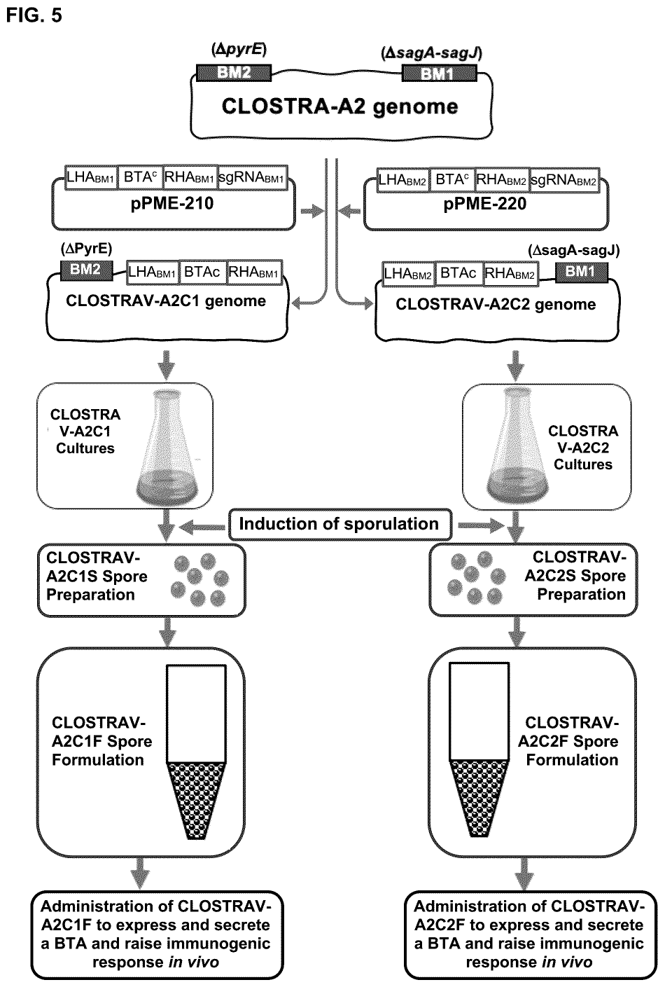

A “CLOSTRAV strain” (or simply CLOSTRAV) refers to a specific example of a CLOSTRA-Aⁿ strain as described in PCT/EP2020/087338 and represented schematically in FIG. 3A (further exemplified in FIG. 4 and FIG. 5), that is a CLOSTRA strain, or CLOSTRA cells, presenting a deleted, inactivated, or at least attenuated, gene that normally provides CLOSTRA with biological activities unsuitable for subsequent uses, in particular where such use of original CLOSTRA causes a decrease in activity, replication, and/or viability of non-CLOSTRA, human cells exposed to CLOSTRA in vivo (or even the properties of CLOSTRA itself when cultured at certain specific conditions and/or to a given density). Thus, the first (or subsequent) HetSeqⁿ present in the Clostra Cassette for generating CLOSTRA-A preferably contains any DNA to be positioned within a Locusⁿ, being a CLOSTRA gene controlling or directly involved in the undesired phenotype, that disrupts, inactivates, substitutes, or otherwise mutates its regulatory and/or coding sequences, but with no or limited effects on CLOSTRA viability and other biological activities. For instance, the first HetSeqⁿ can be a sequence negatively regulating the expression or Locusⁿ , introducing point mutagenesis, partial deletion, insertion or total deletion of the coding region for the complete protein, or purely interrupting and substituting the entire, or a segment of, Locusⁿ with a “bookmark” sequence, without providing any other activity.

As disclosed in PCT/EP2020/087338, the substitution of at least part of an operon involved in haemolytic CLOSTRA activities (Sag operon) with a non-functional, reference, marker, or tag sequence substantially reduces such activity in a CLOSTRA-Aⁿ strain suitable for generating a CLOSTRAV strain. The Locusⁿ and related phenotype functionally inactivated or attenuated in CLOSTRA-A may be also related to antigenic sequences, enzymes, and in general by-products of clostridial metabolism whose secretion and/or accumulation may be undesirable, for instance, including the use of specific molecules (as source of energy or for transcription and/or replication function) and/or the production of acids, alcohols, toxins, or other metabolic by-products.

Moreover, CLOSTRA-A may combine the inactivation or deletion of two or more distinct Locusⁿ following the integration at least multiple, distinct HetSeqⁿ, as shown in FIG. 4A-FIG. 4B and the Examples with CLOSTRA-A1 and CLOSTRA-A2, resulting from sequential use of pPM-1nn derivative plasmids pPME-101 and pPME-105. For instance, the removal or inactivation of Sag operon (as an example of genes coding a toxic activity) can be associated to inactivation or removal of orotate phosphoribosyltransferase (PyrE), orotate mono-phosphate decarboxylase (pyrF), or uracil phosphoribosyltransferase (upp), creating uracil auxotrophs (that is, the inability of an organism to synthesize an organic compound required for its growth, in this case requiring uracil-supplemented medium for growth; Al-Hinai M et al., 2012) with defective hemolytic properties. Such mutants can be also isolated using 5-fluoroorotic acid (5-FOA) or 5-fluorouracil (5-FU), both toxic antimetabolites that are converted to a toxic compound in presence of such enzymes, but, more importantly, present the improved biocontainment of CLOSTRAV-BTA and CBTAS in vivo and also in environments lacking this essential molecule. The auxotrophy feature may be also defined on the basis of minimal growth requirements that are established for a given CLOSTRAV, such as the amino acid (for example cysteine, isoleucine, leucine, proline, or tryptophan) or vitamin (e.g., biotin, pantothenate and pyridoxine) that are required for growth in culture (Karasawa T et al., 1995).

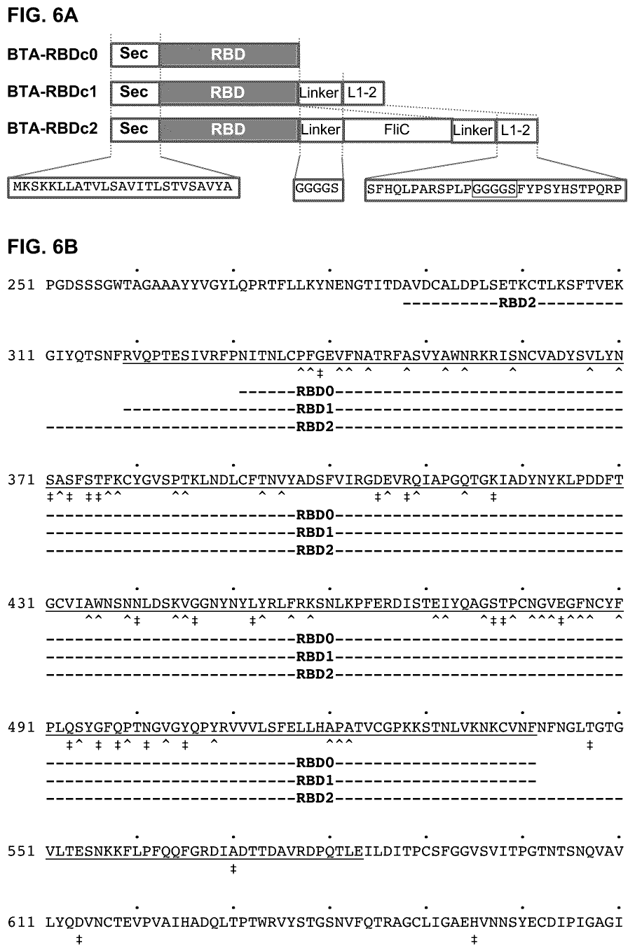

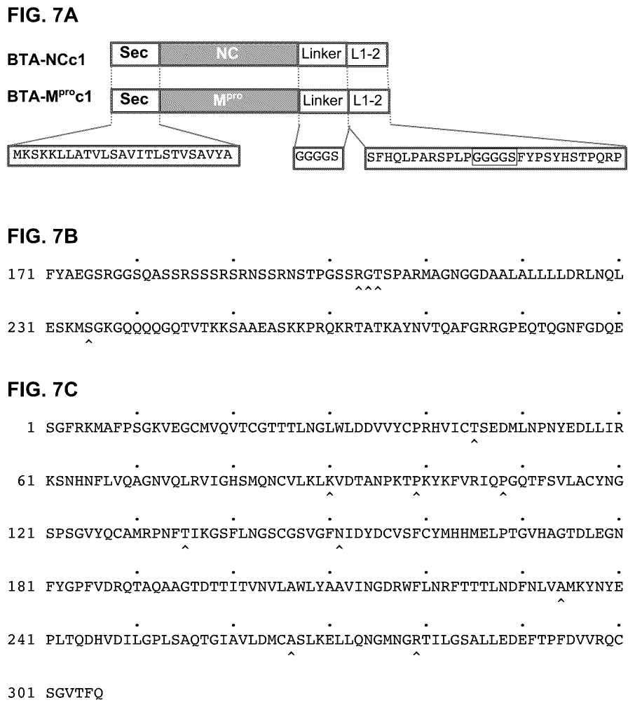

“CLOSTRAV-BTA” refers to a CLOSTRAV strain, or CLOSTRAV cells, presenting a functional heterologous gene coding a recombinant antigen (preferably as a fusion protein) that is not present in the CLOSTRA genome, providing CLOSTRAV-derivative strains that express a protein sequence that, if entering in contact with cells and tissues forming the immune system in mammalian, can elicit an immunological response, in particular against viruses, bacteria, and other pathogenic agents for humans (or animals), to be used as means for vaccination. In a preferred embodiment, the CLOSTRAV-BTA strain is generated by using a pPM-2nn vector comprising a Clostra Cassette and a BTA cassette as schematically represented in FIG. 2B and FIG. 3B. Exemplary arrangements of sequence elements within a BTA cassette and in the reference pPME-200 vector are shown in FIG. 2C and FIG. 3C, respectively, wherein specific series of Clostra Cassettes are designed to express BTA as a recombinant gene in specific CLOSTRAV-BTA genomic locations and to secrete BTA as fusion proteins comprising the recombinant antigen associated to additional, functional protein sequences. The overall process of generating two alternative CLOSTRAV-BTA strains expressing the same BTA (with corresponding CBTAS and CBTAS-F) using the same CLOSTRA-Aⁿ strain is shown in FIG. 5 for two different genomic locations since two alternative pPME-200 vectors are used. Exemplary BTA cassettes including specific sequences in addition to a reference, viral recombinant antigen that can be expressed in alternative CLOSTRAV-BTA strains are shown in FIG. 6A and FIG. 7A.

The CLOSTRAV-BTA strains are characterized by having been modified in at least two distinct genomic locations by distinct Clostra Cassettes but may also combine additional features, as disclosed in PCT/EP2020/087338. In particular a CLOSTRAV-BTA strain may also present an inducible or repressible sporulation phenotype useful for producing CBTAS only in appropriate, controlled conditions during manufacturing and/or after administration of CBTAS-F, so that sporulation of a CLOSTRAV-BTA strain is possible only under specific, suitable conditions for manufacturing or clinical use. The vectors, the targeting sequences, the promoter sequences, and overall strategies for generating CLOSTRA-S strains as disclosed in PCT/EP2020/087338 may be applied to CLOSTRA-Aⁿ, CLOSTRAV, or CLOSTRAV-BTA strains, for instance, by using any of the pPME-104, pPME-106, or pPME-107 vector or any similar vector that can be designed on the basis of the disclosure of PCT/EP2020/087338 that target a sporulation gene in Clostridium species such as Spo0A or SpollAA. Moreover, a CLOSTRA-S or a CLOSTRA-SA strain as defined and generated according to PCT/EP2020/087338 may be used as a CLOSTRAV strain having the desired sporulation, replication, and other biological features for generating CLOSTRAV-BTA strains and producing the related CBTAS preparations.

Additional embodiments apply to general CLOSTRAV-related subject matter under the general definition of “CLOSTRAV-Derived Products”, globally referring to CLOSTRAV strains and cells, CLOSTRAV-BTA strains and cells, spores that are generated by such strains and cells, provided as spore preparations (CBTAS) and formulation for use (CBTAS-F). CLOSTRAV-Derived Products present the functional properties and genomic modification characterizing any CLOSTRAV-BTA, independently from the order in which Clostra Cassettes were used to modify a CLOSTRAV genome. The CLOSTRAV-Derived Products can be provided as purified and/or concentrated preparations or formulations that may be stored or used directly. In particular, due to additional requirements related to use or storage, such preparations may further comprise pharmaceutically acceptable biological or chemical components such as drugs, additives, salts, or excipients. CLOSTRAV-Derived Products can be provided in alternative formats, such as liquid, solid, frozen, dried, and/or lyophilized formats, depending on desired storage, use, or administration.

### (ii) Recombinant Antigens that are Expressed as BTA by CLOSTRAV-BTA Strains

A recombinant antigen whose coding sequence is cloned in BTA^{c} may be the complete or, preferably, a fragment of a protein having immunogenic, immunoreactive properties in mammals, preferably humans. The recombinant antigen is encoded by a DNA coding sequences that may be identical to the one present in the organism where it is naturally expressed but preferably the nucleotides in the codons are optimized for expression in bacteria, and more preferably in Clostridium species. The codon-optimized sequence for the BTA fusion protein may be cloned in the BTA cassette of a pPM-2nn vector, such as any of those pPME-200 derivative strains after having been amplified, re-cloned, and/or synthetically generated using shuttle plasmids or vectors used for generating vaccines in other organisms, including vectors for expressing antigens in E. coli, yeast, and human cells infected with adenoviral vectors.

A recombinant antigen is a protein sequence isolated from a natural protein having any biological properties but preferably the recombinant antigen is either secreted or present on the surface of the non-mammalian, non-clostridial organism of origin (e.g., on the surface of a bacterial cell or viral capsid). The recombinant antigen may be any immunogenic fragment of a protein containing more than 10, 25, 50, 100, or consecutive amino acids. Such protein may have any function (such as structural, human cell- or human protein-binding properties, enzymatic, cytotoxic, pro- or anti-proliferative properties, metabolic, immunological, pro-necrotic or apoptotic, or cell de-/differentiating) that is preferably derived from a bacterium, a virus, fungus, protozoa, plant, archaea or any other non-mammalian organisms that may infect, reside, or otherwise being present in the human body (e.g. within lungs, gut, mouth, genital organs, eye, bone marrow, lymphoid tissues, liver, or stomach) with direct or indirect pathogenic effects (including un desirable allergenic effects) that requires preventive or therapeutic treatments.

The coding and non-coding DNA sequences that are cloned in the BTA cassette to be introduced in CLOSTRAV genome can be identical to those originally disclosed in the literature (either synthetic or natural ones), but they can be modified and adapted to the Clostridium biology and/or of other bacteria where the pPM-2nn vector is generated and used. For example, the codon usage within the coding sequence can be optimized to improve the transcription and translation in Clostridium strains of the corresponding protein, as described in the literature for a series of human or non-clostridial genes that are expressed in Clostridium strain as recombinant proteins. Indeed, specific software can be used for this purpose, such as JCat or Upgene (Gao W et al., 2004; Grote A et al., 2005; Alexaki A., et al., 2019). Similarly, the regulatory sequences that control BTA secretion (as a signal sequence) or expression (for starting or ending transcription) within a BTA cassette should be functional, or at least inducible in a Clostridium strain, and in particular in a CLOSTRAV strain for achieving the desired level of expression and secretion of the recombinant antigen as a BTA fusion protein by CLOSTRAV-BTA cells before and after sporulation. As an alternative to the signal sequence, a sequence that allows expressing BTA as a fusion protein that is immobilized on the external surface of CLOSTRAV-BTA may also be generated.

As observed in one review article (Rappuoli R et al., 2021), improved vaccination means are needed to impede infection, avoid long-term damage, and/or decrease mortality due to new and existing pathogens that, as shown by the ongoing COVID-19 pandemic, are still major health priorities, also because of antimicrobial resistance or chronic infection. This report describes a series of approaches for prioritizing pathogens and vaccination-relevant antigens that may be used for generating the appropriate recombinant antigen for vaccination purposes, which may be expressed (in vitro and vivo) and administered using CLOSTRAV-BTA strains and related CBTAS or CBTAS-F, in particular for blocking the invasion and multiplication of a pathogenic agent (or a combination thereof) in a subject.

The pharmaceutical composition based on a CLOSTRAV-Derived Product as disclosed herein may be preferably used as a vaccine for treating an infection that, as discussed in the review cited above, is appropriate for naïve immune systems or primed immune systems (being primed by controlled, non-controlled, chronic infections). The recombinant antigen may be an isolated protein domain or fragment derived from bacteria, plant (in particular food allergens), fungi, prions, or mycoplasma, in particular those proliferating in specific patients or populations (defined according to age, ongoing treatments for other diseases, genetic features, or other medically relevant criteria) and/or geographical areas since becoming more pathogenic, persistent, and/or resistant to available drugs (such as antibiotics), and in general any agent responsible of infectious or zoonotic diseases. On this latter topic, the literature describes how emerging zoonotic diseases (bacterial, parasitic, viral, Chlamydial, Rickettisial, fungal/mycotic, or protozoal ones) continue to infect humans (or animals) as well as wild and domestic animals and their impact and means for control need to be attentively evaluated, as in the case of orthopoxviruses (monkeypox, cowpox, and vaccinia viruses) or bacterial zoonoses (Silva N et al., 2021; Rahman T et al., 2020).

Specific bacteria and protozoal parasites species or strains of interest for selecting recombinant antigens useful as BTA are found among Escherichia coli, Mycobacteria (e.g. M. leprae or M. tuberculosis), Acinetobacter (e.g. A. baumannii), Staphylococcus (e.g. S. aureus), Streptococcus (e.g. S. pyogenes or pneumoniae), Chlamydia (e.g. C. trachomatis), Klebsiella (e.g. K. pneumoniae), Mycoplasma (e.g. M. pneumoniae), Pseudomonas (e.g. P. aeruginosa), Neisseria (e.g. N. gonorrhoeae), Salmonella (e.g. S. typhmurium, S. enterica, or S. Cholerae), Plasmodia (e.g. P. vivax or P. falciparum), Campylobacter (e.g. E. faecium or E. fetus), Enterococcus (e.g. E. faecium), Borrelia (e.g. B. burgdorferi or B. mayonii, as Lyme disease), Corynobacterium (e.g. C. pseudotuberculosis or C. ulcerans), Rickettisia (e.g. R. australis or R. Rickettsii), Helicobacter (e.g. H. pullorum), Meningococcus, Shigella, Vibrio, Trichinella, Pneumococcus, Echinococcus, Cryptococcus, Fasciola, Ehrlichia, Arcobacter, Actinomyces, Enterobacter, Francisella, Leptospira, Bordetella, or Brucella.

In certain aspects, the recombinant antigen may be derived from viruses, that may be oncogenic, (such as Human Papilloma virus/HPV or Epstein-Barr virus/EBV) or not, in particular those proliferating in specific patients or populations (defined according to age, ongoing treatments for other diseases, genetic features, or other medically relevant criteria) or geographical areas since becoming more pathogenic persistent, and/or resistant to available drugs (such as antivirals). The virus may be any species, strain or other medically relevant variant of cytomegalovirus (CMV), Paramyxoviridae (e.g., Avulavirinae), Orthomyxoviridae (e.g. Alpha-, Beta-, Delta-, or Gammainfluenzavirus), human immunodeficiency viruses (e.g., HIV-1, HIV-2), Coronaviruses (e.g, MERS-COV, SARS-COV-1, and SARS-COV-2), Filoviridae (e.g., Marburgvirus or Ebolavirus), Togaviridae (e.g. Chikungunya virus or Middelburg virus), Flaviviridae (e.g. Dengue, Zika, Japanese encephalitis, West Nile, yellow fever virus, or Hepacivirus such as Hepatitis B/C viruses), herpesvirusdae (e.g. herpes simplex virus HSV-1/-2), Polyomaviridae (e.g. Merkel cell polyomavirus), bunyavirales (e.g, Hantavirdae or Arenaviridae such as Lymphocytic choriomeningitis virus or Lassavirus), Morbillivirus (e.g. Measles virus or canine distemper virus), Enteroviruses (e.g, polioviruses, Coxsackie A/Coxsackie B viruses, and echoviruses), Astroviruses (e.g, HAstV1-V8, Human/VA1-VA4, and strains responsible of gastroenteritis), rhabdoviridae (e.g, Vesiculovirus or Lyssavirus responsible of rabies), Adenoviridae (e.g., human adenovirus A to G), Pneumoviridae (e.g. Metapneumovirus or Respiratory Syncytial Viruses), or Monkey pox viruses (e.g. orthopoxvirus or poxviridae).

The preferred natural antigens for generating the recombinant antigen may be selected from any of those described in the literature as being relevant for the infection and/or pathogenicity and suitable for raising a specific and effective immunological response. Examples of such antigens can be easily identified in the literature such as BZLF1 or Major envelope glycoprotein (gp350) for EBV, hemagglutinin/HA neuraminidase/NA or matrix/M1 or M2 proteins for Influenza virus, or Haemagglutinin for measles virus, ESAT-6 or Rv2654c/TB7.7 for Mycobacterium tuberculosis, hexon or penton protein for Adenovirus (species B, C, E or other causing respiratory tract infections), matrix (M) or hemagglutinin (H) protein for canine Distemper viral, pp65 or Glycoprotein B for CMV, or fusion (F) glycoprotein for Respiratory Syncytial Virus.

The recombinant antigen may be of human (or animal) origin, identified in proteins from normal tissues (e.g., within lungs, gut, mouth, genital organs, eye, liver, stomach, bone marrow, lymphoid tissues, pancreas or brain) or affected by a pathogenic agent or a disease (e.g., a tumour or cells isolated from colon in a Crohn's disease patient, from lung in an idiopathic lung fibrosis patient or from a brain in a multiple sclerosis or dementia patient). The recombinant antigen of human origin may have a direct or indirect pathogenic effects (including an effect stimulating a pathogenic process such as cancer or chronic disease) that requires preventive or therapeutic treatments, including tumour neoantigen or recognized by neoantigen-specific T cell receptors (TCRs) in the context of major histocompatibility complexes (MHCs) molecules that may a critical role in tumour-specific T cell-mediated anti-tumour immune response and cancer immunotherapies. Tumour neoantigens, with or without a viral aetiology, are distinguished from germline and could be recognized as non-self by the host immune system and may derive from nonsynonymous genetic alterations including single-nucleotide variants, insertions and deletions, gene fusions, frameshift mutations, and structural variants. Alternatively, the human protein is expressed at unusually high levels in tissues and organs, leading to pathological consequences (including major or mutated variants if Carbonic Anhydrase IX, CEA, CEACAM6, EpCAM, CD44, TEM1, CXCR4, PD-L1, VEGFR2, PSMA, HHLA-2, B7-H4, HLA-E, CCR8 (Treg), TIGIT, Tie 2, CD44v6, DLL3 embryonic Notch ligand, CD39 CD73, adenosine receptor 2a, EGFRviii, C3, C3a, MCP1, hERG1, CD63, MUCINE 1 TROP2: trophoblast cell surface receptor L, Glycoprotein NMB). The localized administration of BTA including such fragments of human proteins, by means of CBTAS and CLOSTRAV-BTA proliferating in the intestine, may provide an appropriate immunological response in this and other tissues.

In one exemplary embodiment, the recombinant antigen may be isolated from a protein that is present on the surface of a coronavirus. Coronaviruses are single-stranded positive-sense RNA viruses which encodes four structural proteins forming the complete viral particle and are involved in other processes like morphogenesis, envelope formation, budding or pathogenesis: nucleocapsid protein (N or NC), membrane protein (M) and the envelope protein (E) and, most importantly, the spike protein (S) which is a well characterised protein that mediates coronavirus entry into host cells through the fusion of the viral and cellular membranes (Dai L and Gao G, 2021). Examples of Coronaviruses are SARS-COV-2 (severe acute respiratory syndrome coronavirus 2), SARS-COV (severe acute respiratory syndrome coronavirus), and MERS-COV (Middle East respiratory syndrome (MERS) coronavirus), and any other coronavirus that is included in the genera of alphacoronaviruses, betacoronaviruses, gammacoronaviruses, and deltacoronaviruses, eliciting an immunogenic response that allows for blocking the viral infection and/or neutralizing an alpha-, a beta-, a gamma-, and/or a deltacoronavirus, in particular those coronavirus strains capable of infecting humans or animals.

In certain embodiments, the immunological outcome may be specific for a particular genus of coronavirus or for a particular variant or subgroup of a genus, in particular when such variant is associated with a number of mutations in one or more viral proteins that can affect infectivity, recognition by immunological system, symptoms and/or co-morbidities. Through a comparative assessment of SARS-COV-2 sequences in the population, WHO (World Health Organization) has established official definitions for SARS-CoV-2 variants as being a Variant-of-Interest (VOI) or a Variant-of-Concern (VOC), which have been shown to be associated with one or more of the following changes at a degree of global public health significance: increase in transmissibility or detrimental change in COVID-19 epidemiology; increase in virulence or change in clinical disease presentation; decrease in effectiveness of public health and social measures or available diagnostics, vaccines, therapeutics; cause significant community transmission or multiple COVID-19 clusters (as in the variants defined by Greek alphabet letters alpha, delta, Omicron, etc.). The sequences distinguishing such SARS-COV-2 variants that are named and classified according to biological and epidemiological features in the official WHO webpage (https://www.who.int/en/activities/tracking-SARS-COV-2-variants) may be used to establish CLOSTRAV-BTA in which one or more antigens are expressed using the Clostra cassette arrangements as exemplified in FIG. 3C. The positions in Spike (S), Nucleocapsid (NC) and main, 3C-like proteinase nsp5 (M^{pro}) protein that are relevant for designing BTA and corresponding CLOSTRAV-BTA to be used for eliciting an immunological response against one or more VOI or VOC for SARS-COV-2 are indicated within the protein sequences shown in FIG. 6B, FIG. 7B, and FIG. 7C. Additional details on the VOC/VOI nomenclature, their biological characterisation, medical relevance, and distribution in populations of isolated and combined mutations for each SARS-COV-2 protein is presented in the literature (Khateeb J et al., 2021; Mohammad T et al., 2021).

The recombinant antigen of a coronavirus may be comprised in any of the viral S protein, E protein, M protein, or N (NC) protein. The S protein comprises S1-S2 subunits binding to cellular receptors that vary according to the coronavirus species: angiotensin-converting enzyme 2 (ACE2) in SARS-COV, SARS-COV-2 and HCoV-NL63; and dipeptidyl peptidase 4 (DPP4) and aminopeptidase N (APN) in MERS or others alphacoronaviruses. The S1 subunit has two domains: a N-terminal and a C-terminal domain, the latter serving as a receptor binding domain (RBD) for SARS-CoVs being responsible for recognition and binding of cellular receptor, thus becoming the most important candidate for developing recombinant antigens as protein vaccines that would raise a robust immune reaction, with antibodies protecting against SARS-COV-2 infection and thus from COVID-19 (Pollet J et al., 2021). Among them, the spike protein mediates binding to the host's receptor and membrane fusion and is recognized as the most valuable recombinant antigen as vaccine design target. Otherwise, the antigen may be a fragment of an E antigen, M antigen, N antigen, or a combination of fragments from different recombinant antigens (e.g., S+N/NC, S+E+N/NC, etc.). S protein-based vaccines have been described for other Coronaviruses including MERS (WO 2018/115527) or SARS (WO 2010/063685). Specific BTAs can be defined on the basis of these and later disclosures about antigens in Coronaviruses.

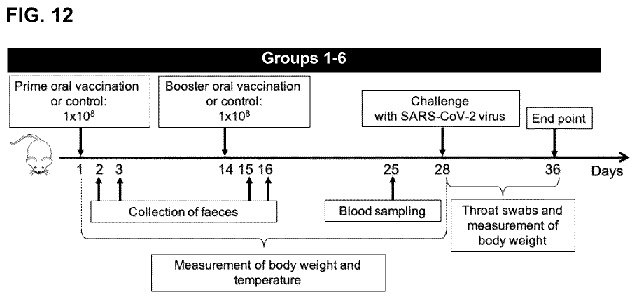

Preferably, the CLOSTRAV-Derived Products elicit an immunological response, and thus can be effectively used as a vaccination means against coronaviruses, by inducing the production of antibodies exhibiting one or more of the applicable preclinical assays such as limitation or inhibition of replication and/or spread in ACE2- and/or TMPRSS2-expressing cells, for instance, in Calu-3 or other cell lines. The efficacy of potential vaccines or antivirals in protecting mice from death or other major effects in various tissues and organs caused by SARS-COV-2 infection may be evaluated in transgenic mice that are engineered to express the human TMPRSS2 and/or ACE2 protein, for example, wherein the mice are infected with an otherwise lethal dose or at least inducing symptoms commonly associated to COVID-19 disease as clinically defined in humans (Caldera-Crespo L et al., 2021. Further evidences may be established in non-transgenic models that have been established in cats, ferrets, hamsters, or non-human-primates and shown to be suitable for proof-of-concept studies for potential vaccine or antiviral candidates (Cleary S et al, 2020). An exemplary validation assay for alternative CLOSTRAV-BTA is provided in Example 2 and FIG. 12, including the opportunity of comparing their effects amongst several biological features.

In the case of SARS-COV-2, the complexity and global impact of SARS-COV-2 requires an attentive study of taxonomy and epidemiology also by studying the genomics, proteomics and molecular features involved in infection and means to overcome the human immune system (Helmy Y et al., 2020; Morens D and Fauci A, 2020). The recombinant antigens within the SARS-COV-2 proteins to be cloned in BTA Cassette and transferred using a Clostra Cassette into a CLOSTRAV strain may be based on protein epitopes that have been mapped for anti-SARS-COV-2 antibodies and compared by activity (such as non-/neutralizing or strain specificities) and reported in the literature about such antibodies (e.g., those known for the literature as CR3022 or VHH72) as summarized in databases like CoV-AbDab (Raybould M et al., 2020) or CoronaVR (Kwong P et al., 2020), or using methods for in silico design or prediction applicable to antibody-guided structure-based vaccine against SARS-COV-2 and other pathogens (Gupta A et al., 2020). The selection of recombinant antigens to be included in a BTA and expressed by means of a CLOSTRAV-BTA may be guided by the analysis of SARS-COV-2 genetic variants distribution across populations and/or geographic area and understanding their epidemiological relevance and virus evolution as suggested in the literature (Song S et al., 2020; Lauring, A and Hodcroft E, 2021) or regularly updated through online databases such as Nextstrain (https://nextstrain.org/ncov/global). Moreover, as summarized in the literature (Caruso F et al., 2020), in silico-defined biomarkers and other means to associate SARS-COV-2 infection (or its (un)successful treatment or prevention) with changes in the gene and/or protein expression in humans, can be used to evaluate candidate therapeutic targets or the efficacy of CLOSTRAV-Derived Products for use in vaccination by bioinformatics analysis.

The recombinant SARS-COV-2 antigen based on the S protein as referenced in the official databases such as UniProt (acc. no. P0DTC2) includes any variant, mutation, or alternative format that is identified in clinical samples or that is in silico designed based on such sequences. Preferably, such variant fragments are those comprised in the sequence shown in FIG. 6B, including those comprising RBD sequences in the S protein fully or partially, optionally including one or more of the mutations that are described in the literature. Other examples of RBD-based sequences that may be cloned and expressed in CLOSTRAV-BTA for vaccination against SARS-COV-2 may be any of those disclosed as being useful for inducing immunization against SARS-COV-2 or eliciting an immunogenic response against the S protein. Exemplary recombinant antigens are provided by full-length or RBD-based sequences from the S protein as disclosed in the literature as useful for preparing vaccines (Wang T et al., 2020; Lu S et al., 2021; CN111518740, CN111375055, CN111333704, CN111732638, CN111518175, CN111217917, CN111228475, RU2738081, US2020407402, US2021000942, WO2021/002776) or associated with, isolated, or combined antibody epitopes from N/NC, E, M^{pro}, and/or S protein (Wang C et al. 2020; Wrapp D et al., 2020; Jakhar R. et al., 2020; CN111848753, CN111393532, U.S. Pat. No. 10,787,501). Alternative RBD-containing, S protein variant sequences may be designed as a recombinant antigen on the basis of glycosylation sites (Yang J et al., 2020), recurrent mutations or deletions in the S protein that would drive antibody escape (McCarthy K et al., 2021; Starr N et al., 2021; Ladner J et al., 2021), stabilized and immunogenic variants (Malladi S et al., 2021), or corresponding sequences in other coronavirus variants that infect humans or other mammalians (Cohen A et al., 2021).

In another embodiment, the recombinant antigen may be a fragment of a protein forming the viral capsid of an influenza virus, preferably, influenza A viruses. Although seasonal influenza vaccines are marketed and are a commercial success, their overall effectiveness is limited and/or variable depending on the prevalent subtype responsible for each influenza outbreak, including the H1N1, H1N2, and H3N2 subtypes. A number of constructs have been established on the basis of HA and NA proteins for developing and manufacturing vaccines across different platforms (Chen J et al., 2020; Wei C et al., 2020). BTA, CLOSTRAV-BTA, and CBTAS can express such protein sequences as a recombinant antigen and may provide an orally deliverable and stable influenza vaccine that could be stockpiled as a pandemic preparedness measure or rapidly expanded from a well-characterized GMP-compliant cell bank (as a CLOSTRAV-BTA or CBTAS-F repository).

The approach described above for defining a recombinant antigen based on a coronavirus-derived protein forming the viral particle can be applied to any of viruses, bacteria, and other pathogenic agents, whose infection and permanence in the human body can be inhibited by vaccination. Moreover, such sequences may be cloned as fusion proteins or in bicistronic vectors expressing immunogenic sequences, e.g., within other SARS-COV-2 proteins (in particular those expressed on the viral surface such as, E, N/NC, S, or M protein) and/or within other viruses or bacteria whose infection may be also present and vaccination may be suitably combined by making use of a single CLOSTRAV-BTA strain. Exemplary constructs and arrangements for such fusion proteins suitable for expression by CLOSTRAV-BTA are shown in FIG. 3C. Moreover, examples of such vaccination systems combining two or more recombinant antigens, one of them being present in S protein of SARS-COV-2 are disclosed in the literature, for instance, by fusing the coding sequence or co-expressing a recombinant antigen from proteins derived from influenza virus HA, NA, OR M1 (see CN111499765, CN111560354, CN111676248).

### (iii) Additional Sequences that Can be Fused to Recombinant Antigens in a BTA

As described above, the BTA preferably contains a signal sequence to be used for secreting BTA by CLOSTRAV-BTA, optionally also when the related pPM-2nn vector is hosted in another prokaryotic host such as E. coli. A series of signal sequences has been characterized as being present in natural (or recombinant) proteins expressed by several Clostridium species (e.g., nprM3, nprM4, cstA1, nprM2, and SH3). Alternative signal sequences suitable for expressing recombinant proteins in Clostridium species and E. coli can be identified in the literature or dedicated databases for defining such sequences (Almagro Armenteros J et al., 2019; Freudl R, 2018 ; Owji H et al., 2018) and corresponding coding sequences may be cloned for their correct expression and activity.

The coding sequence for alternative protein sequences may be cloned between those coding for the signal sequence and the recombinant antigen, or after the one for the recombinant antigen. Independent from the type and function of such alternative protein sequences, the inclusion of linker sequences may be needed to allow the correct structure and flexibility, such as those based on Ser/Gly modules (e.g., GGSGGSGGSGGSGGG, GGGGS, GGGGS(n), or GSG(n) modules) repeated one or more times. Examples of signal sequences and their use for expressing recombinant proteins are provided in the literature (Chen X et al., 2013; Method in Enzymology, Vol. 647, 2021).

CLOSTRAV-BTA may express the recombinant antigen as a fusion protein and can include a protein sequence that improves drug delivery by exploiting biological mechanisms suitable for triggering the immunogenic response after CBTAS-F administration, CBTAS germination, and CLOSTRAV-BTA proliferation in the appropriate tissue or organ. Indeed, the increase in length of both the gene and the translated protein intensifies the metabolic burden on the host, thereby reducing the growth rate and “fitness” of CLOSTRAV-BTA outside the specific, desired location. For example, in the case of mucosal administration (and in the preferred oral administration), BTA should be capable of interacting with any of numerous types of cells and solute carrier transporters throughout the gastrointestinal tract. In particular, microfold (M) cells are present in the follicle-associated epithelium of Peyer's patches or gut-associated lymphoid tissues. Even if constituting a minor fraction of the cells in the intestine, M cells play an essential role in the uptake of antigens and microorganisms since capable of transferring luminal particles and antigens shed from bacteria and viruses to bona fide dendritic cells for antigen presentation. Targeting M cells and (directly or indirectly) dendritic cells (DC) in the intestinal epithelium, the “gateway” to the immune cells of the underlying lymphoid tissue, with high affinity ligands improves efficacy of an oral vaccine.

Among the suitable protein sequences are those included in specific lectins and lectin mimetics, agglutinins, pattern recognition receptor-binding sequences (such as those binding TLR2, TLR3, TLR4, and/or other TLRs included in some bacterial proteins like flagellins), Glycoprotein 2-targeting sequences, or Claudin 4-targeting sequences, complement C5a receptor-targeting sequences, antibodies and various peptidic ligands based on small protein motifs. Details on protein sequences that can target the recombinant antigen to M cells or other cells in the gastrointestinal tract (enterocytes, dendritic cells, Goblet cells, or Paneth cells) or targeting directly dendritic cells, and their suitability for oral vaccination are exemplified and reviewed in the literature (Xu Y et al., 2020; Dillon A and Lo D, 2019; US 2013287810; Owen J et al., 2013).

The sequences coding the targeting sequences may also have adjuvant properties, promoting the induction of a local immune-stimulating environment, such as Flagellin (FliC from Salmonella typhimurium, see UniProt Acc. no. P06179 amino acids 2-495, or from E. coli, UniProt Acc. no. P04949 amino acids 2-498), a potent adjuvant that can trigger local inflammation via toll-like receptor (TLR) 5 (WO2020218829; Hajam I et al., 2017). Moreover, examples of immune co-activators (such as fragments of CD80, CD86, OX40L, CD40, CD137L, Hsp70, or IRAK2) that have been cloned as fusion proteins with recombinant antigens, including those from SARS-COV-2 protein antigens, have been described (CN111588843;CN111533812;CN111494616) and may be adapted to the sequence, cloning strategy, and use of each BTA. Phage display or other peptide-based libraries can be also screened to identify additional examples of peptides and protein motifs for their affinity to these cells and their suitability as Targeting and/or Adjuvant sequences to be integrated in BTAs, as well as for identifying improved adjuvant and/or targeting properties. Indeed, a BTA that is expressed as fusion protein carrying both a targeting domain (a ligand to mediate cellular uptake) and an adjuvant domain (to ensure proper immune activation) would interact with immune cells of the gut in a sustained manner, improving the efficacy of oral vaccination by means of CBTAS-F, eliciting an effective systemic and mucosal immune response.

Those proteins that can be expressed as BTA combining a recombinant antigen (such as one from S protein of SARS-COV-2, and in particular comprising RBD sequence, fully or partially, with or without VOI/VOC-relevant mutations), with human or non-human protein sequences that do not present specific immunogenic properties, but may be useful to improve the presentation or the structure of the recombinant antigen, for example, fragments of immunoglobulin constant region (such as Fc fragment of IgG1 antibody, described in CN111533809 and CN111662389) or a multimerization or oligomerization domain such as ferritin or other bacterial proteins (Powell A et al., 2021; Gwyther R et al., 2019; CN111607002;CN111217918; CN111217919).

### (iv) Vectors and Technologies for Producing CLOSTRAV-Derived Strains

The pPM-1nn plasmids, pPM-2nn plasmids, and related Clostra Cassettes (including any pPME-100 and pPME-200 derivative plasmids) can be produced according to protocols disclosed by PCT/EP2020/087333, and in general those protocols applicable to Clostridium species for generating recombinant variants and the strict requirements for using such biological products in a clinical context, for instance, using the equipment and protocols pertaining to biocontainment, storage, transport, elimination, experimental manipulation, and also uses of genetically modified microorganisms.

As described herein according to the Examples, the disclosed vectors and representative implementing technologies can be adapted for integrating DNA within the genome of an obligate anaerobic microorganism such as CLOSTRA in general (as described in the literature), CLOSTRAV strains and CLOSTRAV-BTA strains. Preferably, such vectors and methods are selected from a CRISPR/Cas system, Cre/Lox system, TALEN system, and homologous recombination-based mechanisms in general. Additional details regarding the disclosed sequences, cloning technologies and assembly of such vectors as a platform can be found in the literature (Nora L et al., 2019).

These methods may optionally comprise use of an exogenous antibiotic resistance gene or other nucleic acid encoding a selection marker conferring a selectable phenotype in CLOSTRAV or CLOSTRAV-BTA strains. The modified selectable marker gene may comprise a region encoding a selectable marker and a promoter operably linked to said region, wherein the promoter causes the expression of the selectable marker encoded by a single copy of the modified selectable marker gene in an amount sufficient for the selectable marker to alter the phenotype in the CLOSTRA-Aⁿ, CLOSTRAV or CLOSTRAV-BTA strain such that it can be distinguished from CLOSTRA strain lacking the modified, selectable (or counter-selectable) marker gene. Such gene may be an antibiotic-resistance gene, a gene encoding a specific metabolic enzyme that utilizes a special nutrient substitute, a gene encoding an enzyme that catalyses a chemical compound to form a distinctive colour, a gene encoding a fluorescent protein, and a gene that encodes a protein with specific affinity for another molecule, heterologous toxin, or an antisense RNA. These markers allow the tracing and the shuttling of the plasmid between the Escherichia coli cloning microorganism and CLOSTRA-Aⁿ, CLOSTRAV or CLOSTRAV-BTA strains, via conjugative transfer from Escherichia coli.

The transformation and genetic modification of CLOSTRAV may involve the replacement of a target DNA sequence (e.g., Locusⁿ) by homologous recombination in the CLOSTRA genome. These technologies comprise transforming the CLOSTRAV strain with a vector comprising an origin of replication(s) permitting its replication in CLOSTRAV and preferably in other microorganism (such as E. coli), and a Clostra Cassette comprising sequences homologous to selected regions around the target DNA sequence. In particular, the CRISPR/Cas9 technology and double-crossover, homologous recombination-mediated chromosomal integration allows the recombination of HetSeqⁿ within the Clostra Cassette and CLOSTRA (or CLOSTRAV) genome, independent from any natural homologous recombination system in Clostridium species. This approach may be performed successively two or more times using appropriate plasmids in a specific or any order, as detailed in the Examples using the exemplary pPME-100 and pPME-200 derived plasmids.

The sequence of promoters, coding sequences and other sequences in the plasmid may be also optimized for the specific CLOSTRA gene expression profile, metabolism and biology, for example, wherein the codon usage of the polynucleotide has been optimized. Alternative promoters can be defined according to the literature (Mordaka P and Heap P, 2018). Moreover, CLOSTRA may also present specific features enhancing frequency and/or efficiency of homologous recombination, due to altered or missing genes involved in CLOSTRA homologous recombination. Typically, homologous recombination is possible due to sequences present in the Left- and Right Homology Arms of a Clostra Cassette that are at least 70%; 80%, 90%, 95%, 99% or more identical to the region downstream and upstream of Locusⁿ within CLOSTRA or CLOSTRA-Derived Products (including specific CLOSTRAV strains), and such homologous sequence contains at least about 50, 100, 250, 500, 750, 100, 1500 bases or more nucleotides.

The site-specific changes in the CLOSTRA strain may involve the use of the Cas9 enzyme (e.g., as identified and cloned in Streptococcus pyogenes and other suitable microorganisms) that may be introduced into the cells using the same plasmid containing the sequences to be introduced in the CLOSTRA genome (e.g., in the pPM-1nn and pPM-2nn plasmids) or by using two distinct plasmids. As used herein, the Cas9 enzyme may exploit one or more DNA sequences that are repetitive sequences associated with the endogenous Clustered Regularly Interspersed Short Palindromic Repeats (CRISPR) or one or more contiguous DNA sequences from the CLOSTRA genome or CLOSTRA-Derived Products (such as CLOSTRAV-Derived Products and CLOSTRAV strains).

The present vector can be introduced into CLOSTRA, CLOSTRAV, and CLOSTRA-Derived Products (such as CLOSTRAV-Derived Products and CLOSTRAV strains) using a DNA delivery technique appropriate for Clostridium species, in particular selected from conjugation, DNA-calcium phosphate co-precipitation, general transduction, liposome fusion and protoplast transformation. In this manner, the Locusⁿ can be modified, wherein the Locusⁿ can be any of the following coding or non-coding sequences (or specific fragments comprised in them): promoters, operons, genes encoding a sporulation factor, a metabolic enzyme, a transcription regulatory protein, a cell growth factor, a toxin, a cell stress response protein, or cell replication factor. Additional details about cloning strategies and alternative sequences applicable to CLOSTRAV and CLOSTRA V-BTA generation are disclosed in PCTEP2020/087338, including with respect to repressible or inducible promoter sequences that can be cloned in the Clostra Cassette and transferred in the CLOSTRAV or CLOSTRAV-BTA strain using a pPM-1nn or pPM2nn vector.

Among the known inducer or repression systems that are active in Clostridium strains and that can allow a proper control levels for expression of any recombinant gene within the Clostra Cassette, two main mechanisms can be exploited. As a main approach, the regulatory sequence may be activated by compounds such as metals, chemicals, or sugars that can be added in cell culture media such as arabinose (Zhang J et al., 2015), lactose (Hartman A H et al., 2011), xylose (Nariya H et al., 2011), or similar, non-native derivative RNA polymerase binding sequences. Alternative, more sophisticated approaches targeting gene transcription, post-transcriptional control or post translational control, may involve the cloning of silencing RNA, specific enzymes, and/or synthetic riboswitches that control gene expression in diverse bacterial species, including Clostridium species, and are based on the use of compounds such as Theophylline and/or lactose that are added in cell culture (Topp S et al., 2010; Cui W et al., 2016; Canadas I et al., 2019). Depending on the CLOSTRAV genome and later manufacturing and use of CLOSTRAV-Derived Products, one or more of such systems can be combined to obtain the desired level of gene expression.

### (v) Pharmaceutical Compositions, Uses, and Methods Involving CLOSTRAV-BTA

The CLOSTRAV-Derived Products can be included in any type of methods, protocol or use where the administration of recombinant antigen may have preventive, prophylactic, or therapeutic use, in particular by raising an immunogenic response against any infectious, parasitic or pathogenic agent (including viruses, bacteria, fungi, contaminants, or allergens present in food, beverage, air, biological samples, or environment). CLOSTRAV-Derived Products can be used for the preparation of vaccines, preferably administered by oral route, that can be manufactured using tablets, capsules, granulates, or other convenient mode for oral administration, in particular by allowing the localized release of CBTAS in the gut where such spores may germinate so that CLOSTRAV-BTA that can proliferate and secrete the recombinant antigen as BTA in anoxic areas where the immunogenic response can be triggered and then exploited against agents present in the same or other tissues. The mode of administration can vary accordingly, with routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal or intra-arterial.

The pharmaceutical composition may comprise CLOSTRAV-BTA for a given pathogenic agent expressing a specific BTA and recombinant antigen, but the composition may comprise one specific CBTAS or a mixture of CBTAS, each permitting the expression and secretion of different BTAs and recombinant antigens (from the same protein, from different proteins of the same pathogenic agent, from different strains or variants of the same pathogenic agent, or even from different pathogenic agents). Multiple CLOSTRAV-BTA strains, expressing different antigens originated from distinct pPM-200 vectors, can be used to generate CBTAS that can be mixed in a “cocktail”. Alternatively, a single CLOSTRAV-BTA strain may express BTA coding sequences for multiple recombinant antigens, either as poly-cistronic Clostra Cassettes or separate monocistronic cassettes that are integrated at different loci, with sequential rounds of integration by means of distinct pPME-200 vectors.

The validation in pre-clinical model of CLOSTRAV-BTA and related CBTAS-F for a given disease should be aligned to regulatory requirements and biology of each pathogenic agent, such as bacteria or virus, in particular with respect to the susceptibility of animals to infectious agents in humans. Using SARS-COV-2 as an example, literature would guide in the choice of such animals for pre-clinical testing, including primates or rabbits (Younes S et al., 2020; Ravichandran S et al., 2020). A vaccine against viruses based on CBTAS-F may be used to treat or prevent infections in specific, more exposed tissues on organs, such as infections in the gastrointestinal tract, in the lower or higher respiratory tract, in sensory organs, or in genitourinary organs. Symptoms of such infections can include high fever, dry cough, gastro-intestinal symptoms such as diarrhoea, organ failure (kidney failure and renal dysfunction), septic shock, and death in severe cases. Additional signs or symptoms secondary to viral infection may be sore throat, taste or hearing loss, muscle or body aches, headaches, infertility or sexual dysfunctions, chest discomfort, shortness of breath, visual disorders, neurological disorders, bronchitis, shortness of breath, and/or pneumonia.

The literature provides additional guidance on the drug formulation technologies that have been adapted for various infections in order to establish non-invasive vaccination regimens that can overcome the harsh gastrointestinal environment and avoid tolerance induction to achieve effective protection. Aside from improvements in oral drug delivery systems based on different targeting strategies (e.g., directed to M cells, as discussed in Section (iii) above), organic or inorganic molecules may be used in preparation of the oral, preferably solid, formulations (such as permeation enhancers, excipients, or carriers), as those already developed to enhance the bioavailability of vaccines and other drugs as described in the literature (New R, 2019; Lal M, 2020; Zheng Z et al., 2018; Vela-Ramirez J et al., 2017). Such technologies may be used to establish CBTAB-F preparations as pharmaceutical compositions, where additional embodiments relating to salts, excipients, additives and doses for CLOSTRAV-Derived Products can be established using methods and compounds described in reference literature such as in Remington's Pharmaceutical Sciences (edited by Adeboye Adejare; 23rd edition, 2020, ISBN: 9780128200070).

Also provided herein are methods for making a pharmaceutical composition comprising a CLOSTRAV-Derived Product, comprising mixing a CLOSTRAV-Derived Product and one or more of a pharmaceutically acceptable adjuvant, diluent, carrier, or excipient thereof, in order to provide appropriate drug delivery systems. Such components can be adapted according to the specific medical indication being treated (e.g., a viral or a bacterial infection), and/or according to the administration means e.g., by injection, by inhalation, topically, or by oral administration. In the latter case, the oral formulation is intended to treat infectious diseases or any other disease affecting the gastrointestinal tract or other tissue or organ where the infectious or pathogenic agent is present, including lung, kidney, blood, liver, sensory organs, heart, bone marrow, lymphoid tissue, or nerves.

An adjuvant that can be used within a CBTAS-F may be selected among the following compounds: a Stimulator of Interferon Genes (STING) agonist; an inflammatory mediator; a RIG-I agonist; an alpha-gal-cer (NKT agonist); a heat shock protein (e.g., HSP65 and HSP70); a C-type lectin agonists (e.g., beta glycan such as Dectin 1, chitin, and curdlan); a TLR agonist (a TLR2/TLR4 agonist such as lipoteichoic acid or lipopolysaccharides; a TLR3 agonist such as double-stranded RNA or poly(IC) molecules; a TLR5 agonist such as flagellin); a TLR6 or a TLR7/8 agonist such as Poly G10 or Resiquimod; a TLR9 agonist such as unmethylated CpG DNA); or any combination thereof.

CLOSTRAV-Derived Products may conveniently be provided in unit dosage forms, whereby such dosage forms can be prepared by any of the methods known in the art of pharmacy and of cell-based pharmaceutical preparations. Administration of the CLOSTRAV-Derived Products as pharmaceutical formulation can be carried out continuously, or according to one or more discrete dosages within the maximum tolerated dose, adapting, as needed, any other drug or standard-of-care treatment, such as anti-viral, anti-bacterial, anti-inflammatory, anti-tumour, with or without the use of adjuvants. Suitable adjuvant to be included in the final formulation (for instance Alum, CpG, Advax, AS03, Manganese, MF59, QS21, TLR ligand, or cytokines such as IL-12, GM-CSF, IL-18, or IL-21) may be selected and adapted according to the preferred route of administration, as reported in the literature for previous vaccines developed against other bacteria or viruses, such as Coronaviruses (Chung J et al., 2021; Liang, Z et al., 2020). The choice of adjuvant tested for vaccines directed to same virus or bacteria, or to species belonging to the same group of agents, for instance those tested in Coronaviruses in the past that may be tested in SARS-CoV-2 vaccines as well (Gupta T and Gupta S, 2020) or for Lactic Acid Bacterial Mucosal Vaccines (Vilander A and Dean G, 2019).

The pharmaceutical compositions according to the invention can contain CLOSTRAV-BTA cells or spores in an amount that is evaluated in terms of biological and/or therapeutic activity, at a calculated concentration of CLOSTRA cells or spores per a given unit, for instance in a range between 10 to 10¹⁵ or more CLOSTRA spores or cells per dose, per ml, or per mg (and typically between 10⁵ to 10⁹ spores). The concentration of CLOSTRA cells or spores may be also defined as a ratio with respect of the concentration of with pharmaceutically acceptable carrier, vehicle, diluent, additives, excipients, solvents, adjuvants, or other compound and drug that is also included in the formulation (e.g. 10⁵-10⁸ spores per mg of an antiviral drug), or alternatively in the format of colony-forming units (CFU) per dose or per kg.

An effective prophylactic or therapeutically effective dose of a vaccine for treating or preventing a viral infection refers to the amount of the vaccine sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. Depending on the severity of the infection, the frequency and the duration of the treatment can be adjusted and the initial dose may be followed by administration of a second or a plurality of subsequent doses of antigens or antigen fragment thereof in an amount that can be approximately the same or less than that of the initial dose, wherein the subsequent doses are separated by at least 1 day to 3 days; at least one week, at least 2 weeks; at least 3 weeks; or more. The composition dosage, in particular with respect to the content of CLOSTRAV-Derived Products (such as CLOSTRAV-BTA spores), can be adapted for each type of administration, regimen, and/or with the administration of one or more additional drugs (e.g., in a combination therapy).

The dosage, regimens and subject selection for administering CBTAS-F or other CLOSTRAV-Derived Product can be adapted to specific medical conditions related to each infectious disease prior and after the actual infection event. For instance, in the case of SARS-COV-2, the regimen and dosage may take into consideration overall COVID-19 and consequent medical effects, including “Long COVID” and post-acute consequences (or pre-existing, aggravating conditions) related to immunological responses, organ or tissue dysfunction (in lung, liver, kidney, brain, or heart), or other chronic, clinical conditions described in the literature (Brodin P, 2021; Amenta E et al., 2020).

A CLOSTRAV-Derived Product can be included as a kit or kit of parts, as a pharmaceutical composition that may be provided as a liquid solution, granulate, or a freeze-dried powder for injection. In particular, when containing CBTAS-F, the kit or kit of parts may also contain a solvent to be mixed with the spores prior to use, wherein the solvent is selected from Ringer's solution, phosphate buffer saline, or other solution compatible with injection in humans. In case of oral administration by means of food or beverage, the final CBTAS-F may be based on the literature for Clostridium and other microorganisms that used as probiotics for improving response against viral infections using ready-to-use preparation (Lopez-Santamarina A et al., 2021). The criteria for pharmaceutical development and validation that have been described in the literature for edible vaccines may be used (Sahoo A et al., 2020). The kit or kit of parts may also comprise another drug or an adjuvant to be co-administered or separately administered. Those skilled in the art using conventional dosage administration protocols can ascertain optimal administration rates for a given set of conditions. Individual dosages of the agents described herein and/or pharmaceutical compositions of the present invention can be administered in a unit dosage form including pre-filled syringes or vials that contain CLOSTRAV-Derived Products at a predetermined concentration and in a therapeutically useful amount. For instance, each unit dosage form may contain from 10 to 10¹⁵ CLOSTRAV-BTA cells or CBTAS, or an alternative amount, for example, from about 0.001 mg to about 1,000 mg of a CLOSTRAV-Derived Product, e.g., preferably about 0.1 mg to about 100 mg, inclusive of all values and ranges there between.

In some embodiments, the agents and/or pharmaceutical compositions described herein may be administered more than once daily, about once per day, about every other day, about every third day, about once a week, about once every two weeks, or about once every three weeks, to be repeated for two or more cycles of administration, using the appropriate delivery methods as described in the literature, in particular for infectious diseases and vaccination (Zhou X et al. 2020). Each cycle comprises two or more successive administrations and/or associated with other regular, standardized, or cyclic therapeutic regimens involving the administration of a further composition comprising a compound (such as antiviral, antiparasitic, antibacterial, immunological therapies, or other state-of-art treatment associated to vaccination) for treating the disease or any symptom of such disease, adapting consequently the regimen, the dosage, and/or the compositions.

### EXAMPLES

### Example 1: Preparation and Preliminary Validation of a Vector Comprising Clostra Cassette Suitable for Expressing and Delivering an RBD-Based Recombinant Antigen Using a Clostrav-BTA Strain and Related CBTAS

### Materials & Methods

### Bacterial Strains, Cell Culture Conditions, and Spore Preparations

The following Clostridium strains, growing at 37° C. in anaerobic conditions, can be used and cultured as described in the literature: C. sporogenes (Wild type or NCIMB 10696; Kubiak A et al., 2015; Cooksley C et al., 2010) and C. butyricum (DSM 10702 or wild type; Tanner R et al., 1981). The following E. coli strains can be used and cultured as described in the literature: TOP10 (Invitrogen; expression or plasmid storage strain, growing at 37° C. in aerobic conditions) and S17-1 (ATCC 47055; conjugative donor strain growing at 30° C. in aerobic conditions). E. coli cells were cultured in LB medium, supplemented where appropriate with chloramphenicol (25 μg/ml) at 37° C. with horizontal shaking at 200 rpm. All anaerobic Clostridium strains were cultured at 37° C. under anaerobic conditions (80% N₂, 10% CO₂, 10% H₂) in a MACS1000 workstation (Don Whitely, Yorkshire, UK) in BFM medium, a solid or liquid medium developed for culturing and obtaining Clostridium cells and spores without making use material of animal origin. Clostridium spores are prepared, purified, and stored according to the literature (Setlow P, 2019). Additional details on culturing conditions, preparation of high titer, and pure Clostridium spore stocks are disclosed in PCT/EP2020/087338 (see in particular Table 1 and 2).

### Vectors and Cloning Strategies

The pATB1C-41 vector derives from pMTL82151 backbone (Heap J et al., 2009) as pPME-100 vectors designated as pPME-101 and pPME-105 in PCT/EP2020/087338. Means to generate other suitable pPME-100 vectors (to be used for establishing CLOSTRAV strains) are also disclosed in PCT/EP2020/087338 and equally apply to pPME-200 vectors (to be used for establishing CLOSTRAV-BTA strains) by re-cloning the BTA cassette from pATB1C-41 vector (where it is functionally validated). PCT/EP2020/087338 discloses the elements in the scaffold of vectors that are used as Gram-positive (pCB102) or Gram-negative (ColE1) replicons, one or more antibiotic selection markers (in particular catP, for selecting plasmid-carrying cells on the basis of chloramphenicol resistance in E. coli and thiamphenicol resistance in Clostridium strains), and at least a transfer gene for the expression of the genes in E. coli that are required for conjugation (e.g. TraJ). This also applies to other cloned or amplified sequences, the cloning strategies, the PCR primers, and restriction sites for generating the Clostra Cassette: the cloning of Cas9 gene from Streptococcus pyogenes (Dep. No. DSM 20565), the primers for amplifying and cloning the correct F2 module (with gene-specific sgRNA, sgRNA scaffold, and promoter for Cas9 expression), the primers for amplifying the sequences to be used as LHA_{pyr}/RHA_{pyr} or LHA_{sag}/RHA_{sag} to generate the F3 element targeting pyrE gene or Sag operon within CLOSTRA genome (respectively), primers and sequences related to BookMark1 and BookMark2 integration, or the primers for amplifying the sequences to be used to generate F3 element targeting Spo0A or SpollAA, making either of them an inducible gene and improving in vivo replication control and biocontainment features (see in particular SEQ ID NO:1 to SEQ ID NO: 17, SEQ ID NO: 44 to SEQ ID NO: 49, Table 3, and Table 4 in PCT/EP2020/087338).

The regulatory sequences in the BTA cassette that can be cloned in the F3 module of Clostra cassette in pPME-200 vectors to control BTA expression are those disclosed in PCT/EP2020/087338 with respect to HetSeqⁿ expression, with reference to fragments within deposited DNA sequences (having their own accession number in the NCBI databases or otherwise referenced in the literature) that are functional in Clostridium and E. coli for specific promoters (thl-s, thl13, thl14, ptb, ptb13, araE, ptb14, fdx, fdx-RsE, fdx13, fdx14, and bgaR-bgaL), terminators (identified as T1, T2, T3, and T4) that are isolated from genomic sequences deposited in databases (e.g. from CP002660.1, associated to C. acetobutylicum DSM 1731, or CP009225.1, associated to C. sporogenes NCIMB 10696; further details are described in Table 5 and Table 7 of PCT/EP2020/087338).

Specific sequences that have been used to amplify, mutagenize, or clone DNA sequences within the vectors identified as pATB1C-41.1, pATB1C-42.1, pATB1C-42.2, pATB1C-42.3, and pATB1C-42.4 have been designed on the basis of corresponding fragments disclosed in PCT/EP2020/087338. Details are provided in Table 1.

TABLE 1

| Name | Sequence | Description |
|---|---|---|
| Pptb-BsaI-F | TACGGGTCTCGTCCATAAGTCAGCAGA | Amplification of the Pptb- |
| (SEQ ID NO: 1) | AAGTATAATGAG | RBD sequence for the |
| | | construction of pATB1C-41.1 |
| RBD-BsaI-R | TATAGGTCTCTGTCTTTAAAAGTTTACG | Amplification of the Pfdx- |
| (SEQ ID NO: 2) | CATTTGTTCTTCACTAGATTAG | RBD sequence for the |
| | | construction of pATB1C-42.1 |
| Lig-BsaI-R | TATAGGTCTCTGTCTTTATGGACGTTGT | Amplification of PfdxnprM3- |
| (SEQ ID NO: 3) | GGAGTTGAATGATATG | RBD-L ad PfdxnprM3-NC-L |
| Pfdx-BsaI-F | TACGGGTCTCGTCCAGTGTAGTAGCCT | (pATB1C-42.2 for example) |
| (SEQ ID NO: 4) | GCGAAATAAGTAAGG | |
| NC-BsaI-R (SEQ | TATAGGTCTCTGTCTTTAGGCCTGAGTT | Amplification of PfdxnprM3- |
| ID NO: 5) | GAGTCAGCACTGC | NC for the construction of |
| | | pATB1C-42.4 |
| pGGv2-R | CTGTTGAACCATTAGCCGTCTC | Amplification of fragments |
| (SEQ ID NO: 6) | | and screening primers |
| pGGv2-F | CCGATCGCGTCTCCTGG | (reverse and forward) for |
| (SEQ ID NO: 7) | | any pATB1C-based vector |
| M13F | GTAAAACGACGGCCAG | |
| (SEQ ID NO: 8) | | |
| M13R | CAGGAAACAGCTATGAC | |
| (SEQ ID NO: 9) | | |

The references to the literature disclosed in PCT/EP2020/087338 for specific experimental protocols also apply, for instance to cell conjugation (Purdy et. al., 2002), validation of haemolytic activity (Totten P et al., 1995), purity and quality evaluation of preparation by light microscopy (Yang W et al., 2009), or growth media for identifying uracil auxotrophic mutants (Lovitt R et al., 1987). The DNA sequences coding for BTA are subject to PCR using specific primers using standard protocols and the amplification products are digested and linearly ligated with appropriate LHA and RHA fragments using Golden Gate assembly cloning system (ThermoFisher Scientific) according to the manufacturer's instructions. The references to the literature, the sequence length and position, tools for designing single guide sequences (sgRNA), and experimental protocols with respect to Cas9-CRISPR protocols disclosed in PCT/EP2020/087338 also apply. Amplification protocols and reagents were carefully selected to maximize the possibility of obtaining a genetically stable strain with the correct plasmid sequence. Phusion polymerase (NEB®) was employed in all PCR cloning reactions due to its extremely high fidelity (>50-fold lower error rate than Taq) and High efficiency Stable competent cells (NEB®) ensured minimal mutation following transformation. Correct coding sequences were confirmed by two Sanger sequencing reactions, covering the forward and reverse strands. DNA sequencing was carried out at every step of strain development. Following conjugation from E. coli S17-1, C. sporogenes transconjugants (as chloramphenicol-resistant E. coli S17 colonies) were sequenced.

The DNA sequence coding for RBD (RBD1) and the signal sequence (nprM3; C. sporogenes strain NCIMB 10696 genomic sequence under acc. no. CP009225.1, range 1,611,069 to 1,611,143) with codon optimization for expression in Clostridium strains is cloned in pATC1C-41.1 (based on plasmids disclosed in Heap J et al., 2009) and put under the control of promoter for Polypyrimidine Tract-Binding (Pptb; C. acetobutylicum DSM 1731 strain NCIMB 10696 genomic sequence under acc. no. CP009225.1, range 1,611,069 to 1,611,143). The sequences that have been selected as the Targeting ligand are disclosed (as such or as functional variants) in the literature as Co1 (microfold cell binding; SFHQLPARSPLP; Kim S et al, 2017) and DCpep (dendritic cell binding; FYPSYHSTPQRP; Curiel T et al., 2004) and were used separately or fused together, separated by a linker sequence (such as GGGGS; L1-2 in FIG. 6A and FIG. 7A).

### Western Blot Experiment

Clones of three independently created RBD-expressing C. sporogenes, the wild-type (WT) parental strain and WT with empty vector were grown for 8 hours. The resultant cultures were centrifuged (7000×g, 10 min) and the supernatant carefully separated from the cell pellet. These two fractions represented extracellular and intracellular proteins, respectively. Cell pellets were lysed (Bug Buster with protease inhibitor) and each fraction was precipitated as described (Schwarz K et al., 2007). The resultant protein suspensions were heat denatured in reducing loading dye (95° C., 10 minutes) and ran on an SDS-PAGE. Separated proteins were transferred from the gel to a nitrocellulose membrane using a Trans-Blot Turbo system. The membrane was blocked (5% dried milk powder) before overnight incubation with mouse monoclonal anti-RBD antibody (RnD Systems prod. MAB105401). Following buffer washes, presence of bound anti-RBD antibody was detected by incubation with HRP-conjugated anti-mouse antibody. Membranes were developed using Tetramethylbenzidine (TMB).

### Results

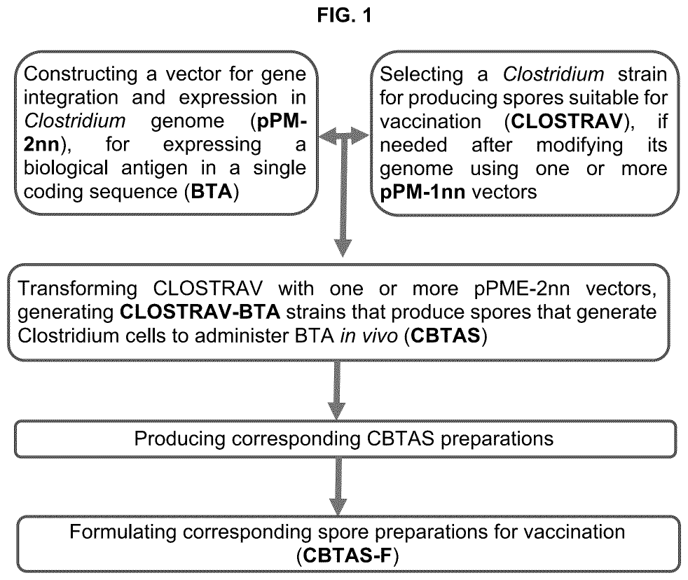

The approach suitable for oral vaccination based on the generation of the CLOSTRAV-BTA strains expressing and secreting a recombinant antigen specific for an infectious agent, can be initially validated using “shuttle” plasmids that are compatible with both Clostridium and E. coli strains and comprise the BTA cassette within pPM-2nn vectors (as described in FIG. 2B and FIG. 3B) with different coding and regulatory sequences to be tested for correct transcription, translation, secretion, and other biological functions using different combinations and arrangements (FIG. 3C). Once validated, the BTA cassette can be re-cloned into an appropriate Clostra Cassette of a pPME-200 vector (as those shown in FIG. 4C) to be used for modified a CLOSTRAV and proceed to the full process for generating and administering CBTAS (as summarized in FIG. 1 and FIG. 5).

This process has been tested using, as an exemplary recombinant antigen, a fragment from the receptor binding domain (RBD) of S protein coded by SARS-COV-2 virus, using the simplest BTA cassette arrangement for expressing a BTA fusion protein (BTA-RBDc0, including only the signal sequence at the N-terminus of the recombinant antigen; FIG. 6A) and an exemplary RBD sequence comprising a major portion of the commonly defined RBD sequence, where also mutations for key SARS-COV-2 variants and alternative fragments are located (RBD0, RBD1, and RBD2; FIG. 6B). The same approach may also be applied to the cloning and expression of recombinant antigens based on the NC protein or M^{pro} protein, as a full or partial protein sequence, and including relevant mutations (FIG. 7).

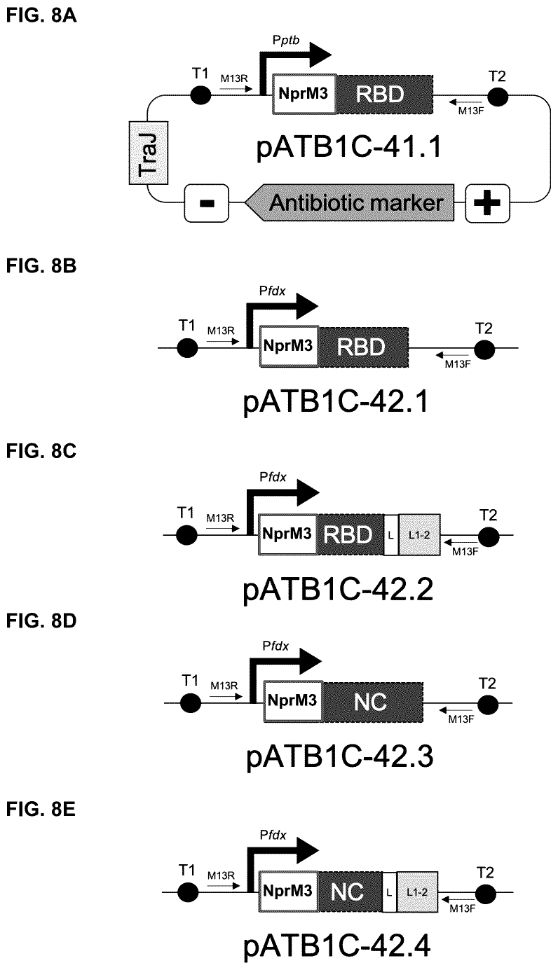

The coding sequence for RBD0, as the exemplary SARS-COV-2 recombinant antigen (BTA-RBDc0), was cloned together with a signal sequence in a shuttle vector termed pATB1C-41.1 (FIG. 8A), which differs essentially from pPME-100 and pPME200 through the absence of elements within the Clostra Cassette that are required for performing the gene transfer into the CLOSTRAV genome (i.e., the LHA and RHA sequences in F3 module and F1/F2 modules). Alternative constructs were established in the same vector wherein the RBD0 and NC recombinant sequences are expressed under a different promoter based on the C. sporogenes ferredoxin gene (Pfdx, associated with the protein coding gene Clspo_c0087; Canadas I et al., 2019) and fused or not with Linker and L1-2 cell targeting sequence; FIG. 6A and FIG. 7A). These alternative constructs are referred to herein as pATB1C-42.1 (FIG. 8B), pATB1C-42.2 (FIG. 8C), pATB1C-42.3 (FIG. 8D), and pATB1C-42.4 (FIG. 8E).

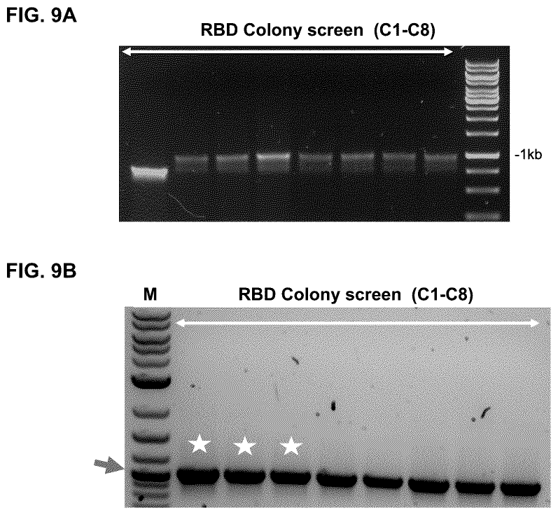

The pATB1C-41.1 vector was initially used to transform E. coli for identifying clones in which the BTA-RBDc0 sequence was correctly integrated in the vector, first by colony PCR screening and then by sequencing (FIG. 9A). The validated clones were used to generate vector preparations to be used for transforming cells from C. sporogenesCLOSTRA-A2 strain (PCT/EP2020/087338) as exemplary CLOSTRAV in which BTA-RBDc0 coding sequence is not integrated but BTA fusion protein can be otherwise expressed and secreted in cell culture medium. Several CLOSTRAV-RBD0 clones contain the vector with the correct sequence (FIG. 9A) and thus were used for further validation.

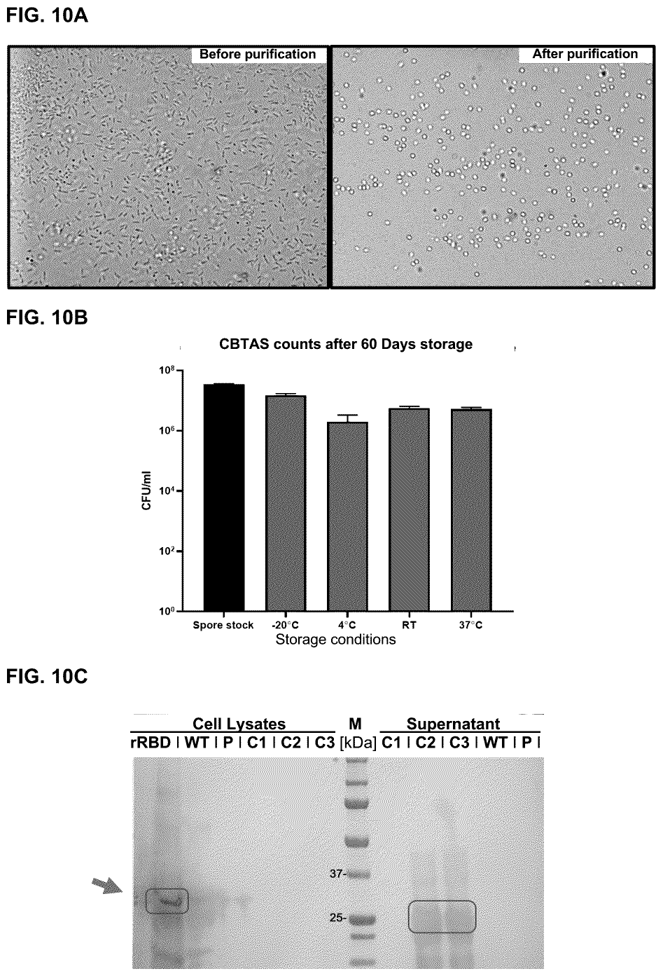

As for any other CLOSTRAV or CLOSTRAV-BTA strain, the influence of different storage solutions and additives on maintaining spore (CBTAS) viability can be tested in CLOSTRAV-RBDc0 strain (i.e. C. sporogenes CLOSTRA-A2 comprising pATB1C-41.1), for instance also regarding the inclusion of compounds in the formulation, such as L-alanine, that are known to improve germination efficiency (Wang S et al., 2017). The CLOSTRAV-RBDc0 strain was not only able to produce viable spores (FIG. 10A and FIG. 10B) but such spores, once germinated, allowed obtaining cells that secreted efficiently BTA-RBDc0, as determined by Western blot assays that confirm that BTA fusion protein is not retained within the CLOSTRAV-BTA cells and is entirely secreted (FIG. 10C). The intracellular and extracellular presence of BTA fusion protein can be also determined by Western blot analysis of cell lysate and supernatant using anti-flagellin antibody.

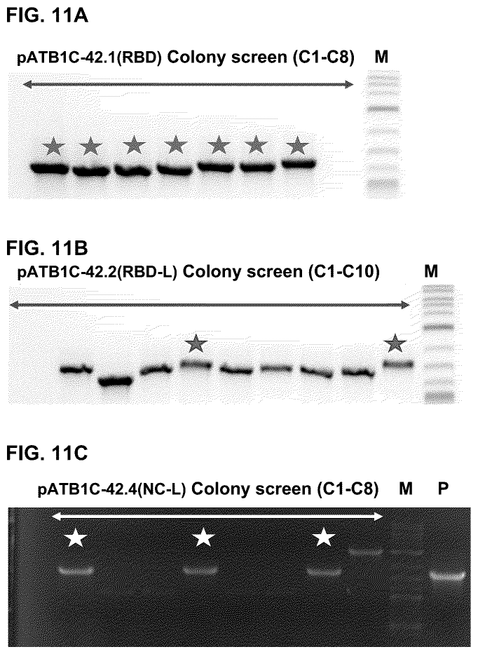

The series of pATB1C-42 vectors shown in FIGS. 8B-8E were constructed and used to generate E. coli clones in which the corresponding RBD- or NC-based are validated as being correctly integrated in the vector, first by colony PCR screening and then by sequencing (FIG. 11). These additionally validated clones were used to generate vector preparations useful for transforming cells from C. sporogenes CLOSTRA-A2 strain (PCT/EP2020/087338), as for pATB1C-41.1 vector.

These data confirm the feasibility of overall approach for generating CLOSTRAV-BTA as disclosed herein, by means of an appropriate CLOSTRAV (e.g. CLOSTRA-A2 strain) and pPM-2nn vector (e.g. pPME-200 and derivative vectors of FIG. 4C) which stably expresses a recombinant antigen that is secreted after the sporulation and germination process after in vivo administration. The corresponding functional CBTAS can be stored, formulated, and used clinically as CBTAS-F, for instance as means for vaccination.

A number of alternative constructs and Clostridium strains can be established as tools for effective vaccination against an infectious agent (such as SARS-COV-2) based on this disclosure and of the disclosure of PCT/EP2020/087338. For instance, a Clostridiumstrain known to sporulate, such as those already characterized for C. butyricum(saccharolytic) or C. sporogenes (proteolytic) can be used since they are natural commensals of the human intestine, with advanced genetic tools for modification available, and having their positive effect on gut and systemic health. The approach disclosed in PCT/EP2020/087338 can be used for generating non-haemolytic, uracil auxotrophic mutants comparable to CLOSTRA-A2 as a reference CLOSTRAV strain. Such platform strain presents the genomic and biological properties suitable for generating panels of CLOSTRAV-BTA strains to be compared functionally according to different integration site of BTA cassette within CLOSTRAV genome (as shown in FIG. 3A and FIG. 4C), different arrangements or combinations of sequences within BTA cassette (as shown in FIG. 3C), different protein and/or DNA coding sequences for a given recombinant antigen (longer, shorter, including immunologically relevant deletions or mutated amino acids), and/or the presence of additional features in the genome (such as integration of further coding or non-coding sequence controlling metabolism, in vivo and in vitro replication, sporulation, or toxicity, by means of pPM-100 vectors).

When this approach is applied to SARS-COV-2 virus and the generation of CLOSTRAV-BTA strains to be used as means for vaccination against COVID-19, in particular by oral administration of CBTAS-F, series of pPME-200 vectors differing at level of integration site in Clostridium genome (see pPME-210/-220/-230/-240 in FIG. 4C) or presenting additional cell targeting or adjuvant sequences (see BTA-RBDc0/c1/c2 in FIG. 6A and FIG. 7A) can be functionally screened for those combining the best safety and immunological profile, using RBD0 (or any variant suggested by literature; FIG. 6B, FIG. 7B, and FIG. 7C) as recombinant antigen. Further studies related to the immunogenicity and vaccination efficacy of SARS-COV-2 antigens that are administered in different platforms (e.g. purified subunit proteins, mRNA- or adenovirus-based vectors) may suggest further constructs comprising RBD and/or other antigen(s) from S protein (e.g. the S2 subunit or the assembly of different epitopes from S1 subunit) as well as from the N/NC, M^{pro}, or E protein. These protein sequences may be inserted into pPME-200 vectors, separately or a fused to each other in order to elicit the desired host immune response. The BTA-RBD coding sequence can be put under the control of a strong promoter, such as Cpe promoter from C. perfringens (Melville S et al., 1994) or any other promoter that, in combination with Adjuvant/Targeting ligands and efficient secretion, can provide CLOSTRAV-BTA strains and related CBTAS adapted to deliver a wide range of BTA-based vaccines based on the in vivo expression of the relevant recombinant antigen(s) within gastrointestinal tract.

### Example 2: Generation and Preliminary Validation of Exemplary Clostridium Strains Combining Non-Haemolytic and Uracil Auxotrophic Properties with BTA Comprising an RBD-Based, SARS-COV-2 Antigen Expression (CLOSTRAV-BTA-RBD)

The validation of a Clostra Cassette described in Example 1 as an approach for expressing a BTA comprising a RBD-based, SARS-COV-2 recombinant antigen using a CLOSTRAV in which the non-haemolytic and uracil auxotrophic properties are introduced by means of the is disclosed in PCT/EP2020/087338 (using pPME-101 and pPME-105 vectors and CLOSTRA-A2 as CLOSTRAV; see FIG. 4A and FIG. 4B). The Clostra Cassette within the pATB vector can be adapted by adding additional components (directly in this shuttle vector or in an appropriate pPM-2nn vector) relevant for raising an efficient immunogenic in vivo, in particular after oral administration in human subjects. The sequence validations and CRISPR/Cas9 protocols that were described in Example 1 and in PCT/EP2020/087338 can be adapted for generating alternative pPME-200 vectors and corresponding CLOSTRAV-BTA in which the BTA is cloned between LHA and RHA sequences that allow targeting such recombinant gene within a preferred location (see the exemplary pPME-210, pME-220, pPME230, and pPME240 Clostra Cassette in FIG. 4C).

Three exemplary arrangements for such BTA are shown in FIG. 6A. All these constructs contain a signal sequence-containing, recombinant SARS-COV-2 RBD based sequence at the N terminus but differ at the C-terminus by the presence or absence of the linker-containing, cell targeting sequence (exemplified by the Linker and L1-2 sequence) that is either directly fused to the RBD sequence or separated by a further element comprising a linker and sequence having adjuvant properties (such as Flagellin C). Regarding the choice of the RBD sequence, the literature indicates a series of alternative sequences based on SARS-COV-2 RBD having slightly different lengths, i.e., longer at N-terminus and/or C-terminus (FIG. 6B). Indeed, these fragments of the S protein contain many of the position where most of mutations found associated to SARS-COV-2 variants having increased infectivity and/or pathogenicity are located. Thus, once the reference RBD sequence is validated as an immunogen suitable for vaccination uses, this sequence may be further mutagenized in one or more positions to generate BTA adapted to populations or geographical areas in which vaccination may require an antigen having a more adequate design and sequence. The CLOSTRAV-BTA strain can be adapted to produce modified antigens according to new variants of the SARS-COV-2 virus that may emerge during future viral outbreaks. Once the DNA or critical amino acid sequence of the new variant is known, the process is advantageously fast and predictable. DNA for new antigen coding sequences can be synthesized, cloned into the pPME-200 integration vector, and conjugated into CLOSTRA. CLOSTRA transconjugants are then screened for integration followed by loss of pPME-200. Following GMP spore production, the new vaccine strain would be ready for clinical use. In addition, the SARS-COV-2/Influenza chimeric vaccine may be provided by combining CBTAS from CLOSTRAV-BTA strains expressing BTA comprising either SARS-COV-2 or influenza recombinant antigens (or a single CLOSTRAV-BTA strain in which the recombinant antigens are expressed in a single BTA fusion protein or by a poly-cistronic Clostra Cassette expressing two or more BTA fusion proteins).

Once that CLOSTRAV-BTA strains expressing RBD variants with specific lengths and/or combinations of sequences (such as those shown in FIG. 6A as BTA-RBDc0, RBD-BTAc1, and RBD-BTAc2, or a sequence identified in any major variants identified in the literature; Lauring, A and Hodcroft E, 2021) are validated at the level of sequence that is introduced in clostridial genome and for general properties (such as replication, sporulation, or confirmation of auxotrophic properties), multiple series of CLOSTRAV-BTA clones for two or more RBD-based BTA variants may be compared using various functional criteria. For example, the level of protein secretion in cell culture conditions can be assessed (as shown in Example 1 using a “shuttle” vector), in normal cell growth condition before or after sporulation, and CBTAS expansion of genetically engineered CLOSTRAV-BTA that secrete BTA inducing mucosal and/or systemic protective immunity. Then, other assays may be related to the safety and viability of CBTAS from such CLOSTRAV-BTA when the spores are released in the environment in soil or water laboratory-controlled conditions, from which samples are extracted at different time points (every week, month, or even less frequently, over 3, 6, 12 or more months) for the recovery of bacterial spores and cultivation in different conditions (anaerobic conditions or different media). In parallel, the CBTAS samples may be exposed to different lyophilisation and formulation protocols to determine which ones would provide CBTAS-F with better properties for later vaccination uses (stability, shelf life, bioavailability, BTA expression levels, etc.).

Comprehensive efficacy and safety evaluations are essential in the development of a vaccine, and we can learn from previous vaccine development programs, for instance those against respiratory syncytial virus, dengue virus, SARS-COV and Middle East respiratory syndrome coronavirus, which highlight the importance of a robust safety and efficacy profile (Su S et al., 2020). Studies and meta-analysis of safety, tolerability, and immunogenicity of presently available COVID-19 vaccines (Speiser D and Bachmann M, 2020; Yuan P et al., 2021) may be used for establishing the most appropriate CBTAS-F-based vaccination schedules. The immunogenicity of BTA produced by CLOSTRAV-BTA strains may be tested by using BTA preparations from CLOSTRAV-BTA or E. coli cell culture supernatants that are injected into BALB/c mice subcutaneously, in the presence of an oil-in-water adjuvant, in a prime-boost regimen with 2 weeks apart. Serum samples are collected and analysed in ELISA for binding to the original recombinant antigen (e.g., full-length HA protein if based on this influenza virus protein or S protein if based on this SARS-CoV-2 protein). In addition, the immune sera can be assessed functionally in rapid, cell-based assay (such as influenza A virus neutralization assay or a SARS-COV-2 S pseudotype virus neutralization assay or T-cell co-culture systems, whereby dendritic cells are fed the vaccine proteins, and co-cultured with T cells, showing their capacity for T-cell activation).

Different in vivo, animal models may be used for preclinical validation of CBTAS and CBTAS-F preparations that contain BTA-RBD coding sequences as a means for raising an immunogenic response useful for preventing SARS-COV-2 infection. In an exemplary set-up for in vivo investigation, distinct experimental groups of mice may be exposed, by oral (intratracheal) or intranasal administration, to formulation media only or to CBTAS-F originated from control CLOSTRAV (CLOSTRA-A2) and different RBD-expressing CLOSTRAV-BTA strains (for example, expressing BTA-RBDc0, BTA-RBDc01, or BTA-RBDc01, including or not one or more relevant mutations). The general effects of each treatment protocol are established, aside from mice viability, using various physiological parameters (such as body weight, biochemical and cell markers in blood, cardiovascular parameters) and behavioural changes (reactiveness, movements) that are regularly measured before administration or after (every week, every other week, or less frequently, over 1, 2, 3, or more months). These data are completed by additional criteria that are measured after culling the mice (at 2, 3, or 4 months) and compared across treatment groups post mortem, such as major changes in size or colour of tissues and organs or other changes within tissue and organs that can be identified only after histopathological analyses. Specific immunological effects of each treatment are established at the intermediate points and the end of experiment in the blood samples that are collected and analysed for specific SARS-COV-2-related criteria: reactive antibodies (specific to the specific BTA-RBD, as determined by ELISA of other more sophisticated in vitro assay for evaluating specificity and affinity of antigen-antibody interaction), markers of immune reaction, specific T-cell activity (the induction of cell-mediated immunity with display of activation markers on immune effector cells) and cytokine profiling. Moreover, faeces obtained at the same time points can be used to identify any specific CLOSTRAV-BTA cell population that is proliferating from CBTAS in the intestines, by measuring bacterial colony forming units under anaerobic conditions or PCR testing and DNA sequencing to detect the presence of nucleic acids originated from CLOSTRAV-BTA specific strains. These analyses may be compared to those similarly performed for determining the presence of CLOSTRAV-BTA in intestines and other tissues obtained post-mortem. The CBTAS-based vaccine vector is not intended to be viable Clostridium cells outside of the patient. The strategy combines passive and active biocontainment systems, in particular based on auxotrophy for uracil production and the need for anoxic environment at appropriate temperature.

Characterising the spatial localisation and temporal persistence of different CLOSTRAV-Derived Products will be an important aspect of vaccine optimisation. Fluorescent reporters have been employed extensively in research and clinical applications, enabling spatial and temporal analysis of biological processes, often in a quantitative manner. Development of a fluorescent reporter capable of functioning in anaerobic environments has proven to be a significant challenge, but several oxygen-independent systems are available and may be adapted to evaluate CBTAS germination and CLOSTRAV-BTA proliferation in vivo, In particular by using adapted Fluorescent In Situ Hybridisation products and methods (Tropini C et al., 2017; Streett H et al., 2013; Seo S et al., 2018).

In the case of CBTAS that are generated and administered as a vaccination means against SARS-COV-2, the CBTAS-F dosage and regimen may be adapted to the combined administration with preventive or therapeutic compositions that are known to be effective against COVID-19. Several clinical trials are in progress, relying on clinical experience, pre-clinical evidence, or computational predictions, in particular using with compounds with antiviral and/or anti-inflammatory activity (Naveja J et al. 2021). In general, this additional therapeutic or prophylactic agent is selected from the group consisting of: an anti-inflammatory agent (e.g. an antibody, such as sarilumab, tocilizumab, gimsilumab, LY-CoV555, 47D11, B38, STI-1499 VIR-7831, or VIR-7832), an antimalarial agent (such as chloroquine or hydroxychloroquine), an antibody or antigen-binding fragment (e.g. specifically binding S protein or human receptors like TMPRSS2 or ACE2). The choice of COVID-19 additional treatments may be based on official, updated guidance from health authorities, as indicated by NIH (Coronavirus Disease 2019 (COVID-19) Treatment Guidelines; https://www.covid19treatmentguidelines.nih.gov/therapies/), NCBI (Emerging Variants of SARS-COV-2 And Novel Therapeutics Against Coronavirus (COVID-19; https://www.ncbi.nlm.nih.gov/books/NBK570580/) by selecting among antiviral agents (such as Molnupiravir. Paxlovid, Remdesivir, or Lopinavir/ritonavir combination), Anti-SARS-COV-2 Neutralizing Antibody Products (Convalescent plasma therapy, Sotrovimab, or antibody cocktails such as REGN-COV2 or Bamlanivimab), or immunomodulatory agents.

One exemplary approach for validating CBTAS-F based on the RBD- and NC-based BTA CLOSTRAV-Derived Products disclosed herein is summarized in FIG. 12. Six distinct treatment groups of golden hamsters, an animal model used for a preliminarily validation of candidate vaccines and as a means to establish effective doses and physiological effects of SARS-COV-2 variants (Cleary S et al, 2020; Ku M W et al., 2021; Abdelnabi R et al., 2021) may be established for evaluating the effects of two distinct constructs for either RBD- and NC-based BTA CLOSTRAV-Derived Products. Depending on the outcome of the animal model, the BTA, the cloning strategy, the vectors, the spore preparation process, the dosage, and/or other feature of the CLOSTRAV-Derived Product may be adapted to improve the efficacy and/or manufacturing of the CLOSTRAV-Derived Product (CBTAS-F) before performing further tests in animal models or in human subjects.

### References

Abdelnabi R et al., 2021. EBioMedicine. 68:103403
Al-Hinai M et al., 2012. 78:8112-8121
Alexaki A., et al., 2019. J Mol Biol, 431:2434-2441
Almagro Armenteros J et al., 2019. Nat Biotechnol. 37:420-423
Amenta E et al., 2020. Open Forum Infect Dis. 7:ofaa509
Brodin P, 2021. Nat Med. 27:28-33
Caldera-Crespo L et al., 2021. Front. Cell. Infect. Microbiol. 11:792584
Canadas I et al., 2019. ACS Synth Biol. 8:1379-1390
Caruso F et al., 2020. Brief Bioinform. 22:701-713
Chen J et al., 2020. J Biomed Sci. 27:33
Chen X et al., 2013. Adv Drug Deliv Rev. 65:1357-69
Cleary S et al, 2020. Br J Pharmacol. 177:4851-4865
Chung J et al., 2021. Adv Drug Deliv Rev. 170:1-25
Cohen A et al., 2021. Science. 371:735-741
Cooksley C et al., 2010. Appl Environ Microbiol. 76:4448-4460
Cruz-Morales P et al., 2019. Genome Biol. Evol. 11:2035
Cui W et al., 2016. Microb Cell Fact. 15:199
Curiel T et al., 2004. J Immunol. 172:7425-7431
Dai L and Gao G, 2021. Nature Rev Immunol. 21:73-82
Dillon A and Lo D, 2019. Front Immunol. 10:1499
Egan M et al., 2021. Gut Microbes. 13:e1863134
Freudl R, 2018. Microb Cell Fact. 17:52
Gao W et al., 2004. Biotechnol Prog. 20:443-8
Grote A et al., 2005. Nucleic Acids Res. 33:W526-31
Gupta A et al., 2020. Front Microbiol. 11:1858
Gupta T and Gupta S, 2020. Int Immunopharmacol. 86:106717
Gwyther R et al., 2019. Biochem Soc Trans. 47:1773-1780
Hartman AH et al., 2011. Appl Environ Microbiol. 77:471-8
Heap J et al., 2009. J Microbiol Methods. 78:79-85
Helmy Y et al., 2020. J. Clin. Med. 9:1225
Hajam I et al., 2017. Exp Mol Med. 49:e373
Jakhar R. et al., 2020. J Med Virol. 92:2114-2123
Karasawa T et al., 1995 Microbiology. 141 :371-5
Khateeb J et al., 2021, Crit Care. 25:244
Kim S et al, 2017. Clin Exp Vaccine Res. 6:15-21
Ku M W et al., 2021. Cell Host Microbe. 29:236-249
Kubiak A et al., 2015. Genome Announc. 3:e00942-15
Kwon S et al., 2020. Front Bioeng Biotechnol. 8:282
Kwong P et al., 2020. Semin Immunol. 50:101428
Ladner J et al., 2021, Cell Reports Medicine 2:100189
Lal M, 2020. Vaccine. 38:4142-4145
Lauring A and Hodcroft E, 2021. JAMA. 325:529-531
Liang, Z et al., 2020. Front Immunol. 11:589833
Lin P et al., 2020. Appl Microbiol Biotechn. 104:2319-2331
Liu G et al., 2022f. Mol Cell. 82:333-347
Lopez-Santamarina A et al., 2021. Foods. 10:130
Lovitt R et al., 1987. J Appl Bacteriol. 62:81-92
Lu S et al., 2021. Cell Reports 34:108666
Ma S et al., 2018. Microb Cell Fact. 17:20
Malladi S et al., 2021. J Biol Chem. 296:100025
McAllister K and Sorg J, 2019. J Bacteriol. 201:e00219-19
Mccarthy K et al., 2021. Science. 371:1139-1142
Melville S et al., 1994. Infect Immun. 62:5550-8
Minton N et al., 2016. Anaerobe 41:104-112
Mohammad T et al., 2021. Front. Cell. Infect. Microbiol. 11:765039
Mordaka P and Heap J, 2018. ACS Synth. 7:672-681
Morens D and Fauci A, 2020. Cell. 182:1077-1092
Nariya H et al., 2011. Appl Environ Microbiol. 77:8439-41
Naveja J et al. 2021. Drug Discov Today. 26:229-239
New R, 2019. Clin Exp Immunol. 198:153-169
Ni G et al., 2019. Biomed Res Int. 2019:1395138
Nora L et al., 2019. Biotechnol Adv. 3:107433
Owen J et al., 2013. Curr Opin Chem Biol. 17:918-924
Owji H et al., 2018. Eur J Cell Biol. 97:422-441
Pollet J et al., 2021. Adv Drug Deliv Rev. 170:71-82
Powell A et al., 2021. ACS Cent Sci. 7:183-199
Purdy D et al., 2002. Mol Microbiol. 46:439-452
Rahman T et al., 2020. Microorganisms. 8:1405
Rappuoli R et al., 2021. Proc Natl Acad Sci U S A. 118:e2020368118
Raybould M et al., 2020. Bioinformatics. 7:734-5
Ravichandran S et al., 2020. Sci Transl Med. 12:eabc3539
Sahoo A et al., 2020. Life Sci. 261:118343
Schwarz K et al., 2007. J Microbiol Methods. 68:396-402
Seo S et al., 2018. J Biotechnol. 265:119-126
Setlow P, 2019. J Appl Microbiol. 126:348-358
Shen S et al., 2019 Microbiol Spectr. 7:10.1128/microbiolspec.GPP3-0017-2018
Silva N et al., 2021. Viruses. 13:43
Song S et al., 2020. Genom Proteom Bioinform. 18:749-759
Speiser D and Bachmann M, 2020. Vaccines. 8:404
Starr N et al., 2021. Science. 371:850-854
Streett H et al., 2013. Cell. 153:1602-11
Su, S et al., 2020. Nature Rev Microbiol. 16:1-9
Tanner R et al., 1981. Current Microbiol. 5 :35-38
Taylor C and Colgan S, 2017. Nat Rev Immunol. 17:774-785
Topp S et al., 2010. Appl Environ Microbiol. 76:7881-4
Torres W et al., 2018. J Cancer Metastasis Treat. 4:4
Totten P et al., 1995. Infect Immun. 63:4409-16
Tummala S et al., 1999. Appl Environ Microbiol. 65:3793-3799
Tropini C et al., 2017. Cell Host Microbe. 21:433-442
Vela-Ramirez J et al., 2017.Adv Drug Deliv Rev. 114:116-131
Vilander A and Dean G, 2019. Vaccines. 7:150
Xu Y et al., 2020. J Control Releas. 322:486-508
Yang J et al., 2020. Nature. 586:572-577
Yang W et al., 2009. J Appl Microbiol. 106:27-33
Younes S et al., 2020. Rev Med Virol 18:e2196
Yuan P et al., 2021. SSRN.3746259
Wang C et al. 2020. Nature Comm. 11:2251
Wang S et al., 2017. Front Microbiol. 8:2047
Wang T et al. 2020. Int J Biol Macromol 160:736-740
Wang Y et al., 2018. Infect Immun 86:e00489-18
Wei C et al., 2020. Nature Rev Drug Disc. 19:239-252
Wrapp D et al., 2020. Cell. 181:1004-1015
Zhang J et al., 2015. Biotechnol Biofuels. 8:36
Zheng Z et al., 2018. Hum Vaccin Immunother. 14:1717-1733
Zhou X et al. 2020. ACS Nano. 14:12370-12389

1. A genetically modified Clostridium strain expressing at least one recombinant antigen and presenting one or more additional genome modifications for deleting and/or to inactivating at least one Clostridium gene, wherein said strain is modified to inactivate or attenuate:
a) at least one Clostridium gene encoding a toxic activity; and
b) at least one Clostridium gene encoding a metabolic activity.

2. The genetically modified Clostridium strain according to claim 1, wherein the toxic activity is haemolysis.

3. The genetically modified Clostridium strain according to claim 1, wherein the metabolic activity is the ability to synthesize uracil.

4. The genetically modified Clostridium strain according to claim 1, wherein the Clostridium strain is modified to present an inducible or repressible sporulation phenotype.

5. The genetically modified Clostridium strain according to claim 1, wherein the recombinant antigen is expressed as a fusion protein comprising a signal sequence for secretion, a cell targeting sequence and/or an adjuvant sequence.

6. The genetically modified Clostridium strain according to claim 5, wherein said fusion protein comprises a signal sequence for secretion and a sequence targeting the recombinant antigen to M cells.

7. The genetically modified Clostridium strain according to claim 1, wherein the genetically modified Clostridium strain expresses at least two recombinant antigens in a one, two, or more distinct fusion proteins.

8. The genetically modified Clostridium strain according to claim 1, wherein the recombinant antigen comprises an antigen expressed by a virus or a bacterium.

9. The genetically modified Clostridium strain according to claim 8, wherein the virus is a Coronavirus or influenza virus.

10. The genetically modified Clostridium strain according to claim 9, wherein the Coronavirus is SARS-COV-2 and the antigen is comprised in the Spike (S) protein sequence or Nucleocapsid (NC) protein sequence.

11. The genetically modified Clostridium strain according to claim 10, wherein said antigen comprises a sequence having at least 90% identity with RBD1 protein (SEQ ID NO: 11), RBD1-L fusion protein (SEQ ID NO: 12), RBD0 protein (SEQ ID NO: 13), RBD0-L fusion protein (SEQ ID NO: 14), RBD2 protein (SEQ ID NO: 15) or RBD2-L fusion protein (SEQ ID NO: 16).

12. The genetically modified Clostridium strain according to claim 11, wherein said antigen is expressed using the DNA sequence PptbnprM3-RBD1 (SEQ ID NO: 28), PfdxnprM3-RBD1 (SEQ ID NO: 29) or PfdxnprM3-RBD1-L (SEQ ID NO: 30).

13. The genetically modified Clostridium strain according to claim 10, wherein said Nucleocapsid (NC) protein sequence comprises a sequence having at least 90% identity with NC protein (SEQ ID NO: 17), NC-L fusion protein (SEQ ID NO: 18), LKR region of NC protein (SEQ ID NO: 19), or LKR-L fusion protein (SEQ ID NO: 20).

14. The genetically modified Clostridium strain according to claim 13, wherein said antigen is expressed using the DNA sequence PfdxnprM3-NC (SEQ ID NO: 31) or PfdxnprM3-NC-L (SEQ ID NO: 32).

15. The genetically modified Clostridium strain according to claim 1, wherein the Clostridium strain is a derivative from the group of Clostridium species comprising C. butyricum or C. sporogenes.

16. (canceled)

17. (canceled)

18. (canceled)

19. (canceled)

20. (canceled)

21. (canceled)

22. A method for preventing or treating a disease comprising administering a pharmaceutical composition according to claim 26 to a subject in need thereof.

23. The method according to claim 22, wherein the disease is an infectious disease.

24. The method according to claim 23, wherein the infectious disease is COVID-19.

25. (canceled)

26. A pharmaceutical composition comprising cells from the genetically modified Clostridium strain according to claim 1.

27. The pharmaceutical composition according to claim 26, wherein the pharmaceutical composition is a vaccine.
